(19) United States
(12) Patent Application Publication
(10) Pub. No.: US 2008/0274495 A1
(43) Pub. Date: Nov. 6, 2008

(54) Diagnostic Method for Testing Hydration and Other Conditions

(75) Inventors: Julie Kemp JUMONVILLE, Austin, TX (US); Myron C. RAPKIN, Indianapolis, IN (US); Julius GOEPP, Rochester, NY (US); Lisa WILLIAMSON, Austin, TX (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: UpSpring Ltd., Austin, TX (US)

(21) Appl. No.: 12/115,312

(22) Filed: May 5, 2008

Related U.S. Application Data
(60) Provisional application No. 60/924,246, filed on May 4, 2007.

(51) Int. Cl. C12Q 1/24; G01N 1/28

(52) U.S. Cl. 435/30; 436/180

(57) ABSTRACT

Methods for the non-invasive collection of a liquid sample in small quantities, such as urine for testing hydration in infants and incontinent adults, directly at the source of that sample, isolation of the collected sample from a testing site, the transportation of that sample to a site for testing contained within the device itself, and the selective application of some or all of the specimen to a testing apparatus contained within the device itself, with a minimum of specimen handling required. Other aspects include a method and device for facilitating the reading of the result of the specimen test by a skilled or unskilled user, including means for determining that no adequate sample has been delivered to the testing site.

### CROSS-REFERENCES TO RELATED APPLICATIONS

This application claims the benefit, under 35 U.S.C. § 119(e), to Provisional Patent Application No. 60/924,246 filed on May 4, 2007, which is hereby incorporated by reference in its entirety herein. This application is related to U.S. patent application Ser. No. ______, entitled “Diagnostic Device for Testing Hydration and Other Conditions” attorney docket no. 027414-000210, which was filed simultaneously with this application on May 5, 2008, and is incorporated by reference herein, in its entirety.

### FIELD OF THE INVENTION

The invention relates generally to collection of body liquids for human and animal diagnostics to test for hydration or other conditions and, more particularly, to the collection and testing of liquids from sources in which volumes of liquids suitable for testing may be limited in quantity or difficult to obtain, e.g., urine specimens from humans who are unable to cooperate in the urine collection process (for example, infants in diapers and incontinent adults).

### BACKGROUND

Liquid testing, specifically testing of urine specimens, is an extremely information-rich diagnostic methodology that is widely used in healthcare and other fields. However, there is much need for improvement in the collection and testing of urine and other bodily liquids that may be difficult to obtain directly from their source and/or which may be available in only very small quantities. Collection and testing of liquids, regardless of the source and ultimate test to be conducted, typically requires the following general steps:

1) Collection of the liquid from the source into an appropriate container that minimizes degradation of the specimen or helps to maintain specimen quality;

2) Presentation of the liquid specimen to the site at which the testing will be conducted;

3) Application of some or all of the specimen to a testing apparatus that undergoes a change as a result of its interaction with the specimen;

4) Reading and recording of the result of the testing process either by a human tester or by an automated or semi-automated device;

5) Reporting of the test result to an individual with skills and knowledge sufficient to take appropriate action as a result of the specific information provided by the test;

6) Interpretation of the test result by that individual, alone or in collaboration with the subject of the test or a party responsible for the subject of the test; and

7) Acting on the results of the test, again either by an individual with skills and knowledge in the field of the test's results, or by such an individual in collaboration with the subject or the subject's responsible party.

Regarding Collection Step 1 above: In actual practice and as applied, for example, to the collection of urine for testing, Step 1 is performed by the individual who is the subject of the test, by means of collection of a substantial volume (more than a few cubic centimeters) of urine in a cup or similar container. Alternatively, Step 1 may be conducted by a health care professional by means of an invasive technique such as the placement of a catheter into the urinary bladder, or by direct puncture of the bladder with a needle and aspiration of urine through the needle. An additional alternative is the placement of a urine collection bag which is affixed to the skin surrounding the urinary meatus (opening through which urine is released); the bag then passively collects urine when it is produced.

All of these existing means of collecting urine present problems when the subject is unable to actively participate in the collection process (e.g., is an infant or incontinent adult). Collection into a specimen container is impractical, while the invasive techniques of catheterization and aspiration are painful and pose risks of injury and infection. Urine bags are notoriously unreliable, frequently leaking the collected specimen before it can be retrieved. Removal of the bag from the skin in the genital region is also painful and distressing for family members to observe. None of these methods is currently in routine use in the home environment; as a result, the collection of urine for home testing of any kind represents a technology that is essentially unavailable to infants, incontinent adults, and their non-skilled caregivers.

Regarding Presentation Step 2 above: In actual practice and as applied, for example, to the presentation of urine for testing, Step 2 is performed by the physical movement of a specimen container holding the specimen from the site of collection some distance to the site of testing. Most commonly this step involves movement of the container from the patient's bedside to a hospital laboratory; in primary care and emergency department settings “near patient testing” may be conducted within a much shorter distance but still not immediately adjacent to the subject of the test.

Transportation of the specimen over such distances presents opportunity for a number of undesirable events to occur prior to actual testing of the specimen. Infection risk to the caregiver, contamination, adulteration, or other alteration of the specimen is increasingly likely as the distance and time between collection and testing increases. In addition, opportunities for errors in specimen identification multiply with time, distance, and the number of individuals who handle the specimen along the way. Direct self-testing of urine eliminates many of these constraints and is available for numerous tests, but none of these are suitable for use in individuals who cannot provide a volume of liquid urine for testing.

Regarding Application Step 3 above: In actual practice and as applied, for example, to the application of urine to a device for testing, Step 3 is performed in a healthcare facility by a skilled individual who is trained in the performance of the test; alternatively, in the case of home urine testing devices, urine is applied to the test device directly by the patient or the patient's caregiver.

Application of liquid to the testing device offers additional opportunities for use errors, contamination, and other events that can adversely affect the outcome of the test. A user must assure the delivery of at least a drop of liquid specimen onto the testing device (in the case of a test strip). This degree of handling of liquid specimens offers opportunity for contamination, adulteration, spillage, and incorrect test results, for example. Again, all such existing means for urine testing require the application of a liquid quantity of the specimen to be tested, and as such are unavailable for use in individuals who cannot produce a liquid sample in a controlled fashion, such as those wearing diapers.

Regarding Reading and Recording Step 4 above: In actual practice and as applied, for example, to the reading and recording of urine test results, Step 4 is performed either by a skilled individual who is trained in the performance of the test or, in the case of home testing devices, by the individual whose liquid is being tested or that person's caregiver. Output of results from testing devices is provided in many different forms; in automated analyzers output may be produced as electronic information that may be transmitted or printed, while in simpler qualitative or semi-quantitative testing the results may be produced in the form of a color change or visible change, appearance, or disappearance of a symbol inherent in the device.

When a result output is automated and/or electronic, it may also be automatically recorded and/or transmitted to the individual who will interpret the result. Most tests for home use, however, produce a visual signal that must be subjectively read by the individual performing the test. Such tests are typically provided with a reference legend (e.g., a color chart) that indicates the approximate value of each interval of visible change that is produced. Users must therefore compare the actual result with the reference chart, which introduces opportunities for error and imprecision in reading results. Yet again, all such existing means for reading and recording of test results are unavailable for use in individuals who cannot produce a liquid sample in a controlled fashion, such as those wearing diapers.

Regarding Reporting Step 5 above: In actual practice and as applied, for example, to the reporting of urine test results, Step 5 is performed by the individual who performs the test. Such reporting may be conducted in real time or in the form of regular, scheduled release of result information. Alternatively, in the case of many home testing devices, results are simply recorded in a home log (or not recorded at all), and only reported to a healthcare professional when abnormal or at regular intervals.

Reporting of test results provides another opportunity for the introduction of error, especially when results are subjective in nature, and/or when at least one result indicates an apparently “normal” value. In home testing conditions, such an apparently “normal” value may not be reported at all. Because no single test provides comprehensive information about a health condition, lack of reporting any result, even a normal one, may result in an adverse outcome. Yet again, all such existing means for reporting of test results are not applicable for use in individuals who cannot produce a liquid sample in a controlled fashion, such as those wearing diapers.

Regarding Interpretation Step 6 above: In actual practice and as applied, for example, to the interpretation of urine test results, Step 6 is performed either by a healthcare professional who is presented with the results, or, in the case of home testing, by the individual who performed the test. Depending on the specific nature of the test and its result, such interpretation may require a high degree of understanding of the principles and practice of medicine; alternatively, it may require only the ability to understand the immediate test result in the narrow context of the disease process under consideration (for example, the meaning of a positive test for glucose in the urine).

Interpretation of test results without direct and timely communication between the individual performing the interpretation and the individual affected by the result offers additional opportunity for error and adverse outcomes. Delayed interpretation can mean loss of an opportunity to intervene and interrupt a disease process early in its course. Interpretation by the patient or patient's unskilled representative poses the risk of incorrect or biased interpretation based on a host of physical and psychological factors entirely unrelated to the test (for example a reluctance to undergo further testing or other procedures). Yet again, all existing means for interpreting test results are unavailable for use in individuals who cannot produce a liquid sample in a controlled fashion, such as those wearing diapers.

Regarding Action Step 7 above: In actual practice and as applied, for example, to acting on the results of urine tests, Step 7 is performed either by a healthcare professional who is presented with the results, or, in the case of home testing, by the individual who performed the test. Appropriate actions may range from doing nothing to immediate presentation to a health care facility. Alternatively, appropriate actions may include administration of therapies in the home setting, with subsequent re-testing and completion of the other steps.

The ability to take informed action based on the result of a simple test performed at home represents one of the major advantages of home testing methodologies, and such tests have a well-established record of both early identification of potentially ominous developments in health status, and also at reduction in unnecessary utilization of health care resources when test results are reassuring or permit institution of a relatively low-technology intervention at home. One of the best-known interventions of this kind is the use of oral rehydration therapy (ORT) for individuals with diarrheal illness at risk of becoming dehydrated; ORT is credited with saving millions of lives annually. Published criteria for initiation of ORT include an assessment of hydration status, and such assessment is available in the form of a home dipstick that measures urine specific gravity as an indicator of hydration status. Unfortunately, all known means for acting on test results in such a fashion are unavailable for use in individuals who cannot produce a liquid sample in a controlled fashion, such as those wearing diapers (and in the case of the example given here, such individuals represent up to 50% of people in whom such testing might be considered).

In summary, a need exists for a diagnostic method and device that allows the performance of all of the preceding 7 steps in a simple, controlled, and easily-used system that reduces or eliminates many of the opportunities for errors described above. Most importantly, no method or device currently exists for the collection of small quantities of urine from diaper-wearing individuals and direct introduction of that specimen to a testing apparatus. The absence of such a method and device makes a variety of urine tests virtually unavailable in infants and other diaper-wearing individuals, resulting loss of potentially valuable health status information, or, alternatively, in the use of other, more invasive forms of testing, or, yet again, in the potential application of unnecessary forms of treatment in the absence of such health information. In this application, therefore, we disclose such a diagnostic method and device with particularly advantageous application to the collection of urine and other liquids that are similarly difficult to collect, transport, and test.

Turning now to the evaluation of hydration status specifically, many means exist for determination of hydration status based on evaluation of urine characteristics. The most common of these is determination of urine specific gravity (SG), which is a measure of the concentration of urine (the amount of solid material dissolved in a given volume of urine). Urine concentration is determined by the kidney's response to circulating fluid volume, such that as circulating fluid volume decreases (in the case of dehydration, for example), urine concentration is increased as a means of retaining water for circulation. The clinically meaningful range of urine specific gravity is established to lie between 1.000 (the SG of water) and about 1.030 (a highly-concentrated urine).

The clinical reference standard for SG determination is “Light” or “Optical” refractometry, which exploits the differences in refraction of an incident beam of light by liquids of differing specific gravities.

Light refractometry is fairly easily performed by a skilled individual, but the equipment is costly and must be maintained and calibrated consistently.

Dry-phase chemically-treated reagent papers (e.g. dipsticks) are in widespread use for estimation of urine specific gravity, which for most urine specimens is a reliable and valid surrogate measure of SG. Reagent dipsticks produce a color change when exposed to a liquid specimen, and the resulting SG is read from a color chart typically containing a range of possible colors representing SG values in the clinically meaningful range.

Dipsticks are easily used and require minimum training to read results, but they require at least a single drop of liquid urine to activate the test. Individuals who can cooperate in the test are able either to collect a specimen in a container, or to urinate directly onto the dipstick to produce a result. Modern highly-absorbent diapers, however, retain urine so tightly within their absorbent material that insufficient urine can be expressed from the diaper to produce a valid test result. Additionally, the polymer gel formed by urine in contact with the absorbent material has ion-trapping properties, changing the ionic strength (and hence the indicated SG) when a dipstick is used. Finally, the set of chemical reactions that takes place in the dipstick is inherently sensitive to the pH of the test liquid, such that indicated SG is lower than true SG at high pH values. This pH sensitivity is a potential source of at least minor errors in SG determination by this method.

As a result of these various limitations in existing methodologies, the determination of urine SG, a valuable contributor to the evaluation of hydration status, is essentially unavailable in the large population of people who would benefit the most from its use, for example, infants and other diaper-wearing individuals with diarrheal illness who are at risk for dehydration. The problem is not trivial: dehydration from diarrhea remains the single largest cause of death in children under 5 years of age in the world. In the United States alone roughly 20 million episodes of diarrheal illness occur annually in children in that age group, with an estimated cost for health care services of between 1 and 2 billion dollars.

In summary, a need exists for a more effective method and device that makes determination of urine specific gravity, a valuable contributor to the evaluation of overall hydration status, available particularly to the large population of individuals who cannot participate directly in the collection and testing of urine for this purpose. In this application we disclose more effective diagnostic methods and devices that enable specific gravity testing of urine from diaper-wearing individuals; the methods and devices overcome the difficulties associated with collection in this population as outlined above and in the prior art review outlined below, including preventing the contact of tested urine with diaper absorbent material, providing a means for introducing a pH correction such that the inherent pH sensitivity of the reagent test is mitigated, as well as other novel diagnostic and customer use features described herein.

Numerous types of devices and methods have been proposed for body liquid collection and analysis, but a practical, commercially viable approach to the problems discussed above, has not been found, as the following review of certain types of prior art devices and methods demonstrate. For example, U.S. Pat. No. 5,468,236, to Everhart et al. discloses the use of an absorbent product which includes a chemically reactive means having an end point adapted to provide a visual indication of the presence of a substance in mammalian bodily excrement. The visual indication occurs as a result of interaction with the mammalian bodily excrement. The use of a chemical reactive means that is integrated in a functional disposable (e.g. diaper) creates a number of problems in this approach. For example, the need to visually read the same surface of the chemically reactive means that is exposed to excrement provides no protection for the chemical reactive means against additional chemical interferences that exist within the excrement. The Everhart et al. device allows the excrement to contact the same surface of the chemical reactive means that is visually inspected, which provides no protection against the optical interferences that exist within the excrement, such as optical reflectance, optical refraction, or turbidity, which can unpredictably and adversely affect the optical characteristics of the surface of the chemically reactive means and the proper interpretation of the results of the chemically reactive process. The Everhart et al. device may also create a health/biohazard risk for the patient. Because the chemically reactive means are integrated in the sample collection means, the device requires that chemical reagents be placed into direct contact with the skin of the patient, with the resultant potential for adverse reaction with the skin. The ability of all excrement to contact the integral chemical reactive means, for example, makes it possible for a urine-based test to be activated by fecal matter, and vice-versa, without being apparent to the user, which could result in incorrect diagnosis and treatment. The Everhart et al. device does not provide feedback to the user to identify this potentially dangerous condition.

U.S. Pat. No. 6,673,630 to Albarella et al. discloses the visual use of urine test strips and reagent pads by providing a single color from a reference color spectrum directly on the calorimetric strip and adjacent to the reagent area to allow easy comparison. According to this patent, after a fluid has been applied to the reagent strip, a technician can compare the color of the reagent area to reference colors to determine presence of a substance in the fluid. Although Albarella et al. attempt to increase the ease of analysis of fluids, there are still numerous problems related to the use of test strips and reagents as discussed below.

The small size of the reagent area on the Albarella test strip requires precise alignment between the fluid transfer device and reagent area. This positional sensitivity makes the Albarella device prone to user-related errors. The juxtaposition of reference colors next to the reagent pad allows for possible seepage of fluid from the reagent strip to the reference colors. Because sample fluids have optical properties such as reflectance, refractive index, color and/or opacity, it is possible for these fluids to alter the appearance of the reference colors and jeopardize accurate interpretation of the results produced by the activated reagent pad. Albarella's device interprets the activated reagent strip from the same surface that receives the sample fluid. This provides no protection against optical interferences, such as variations in the optical reflection, refractive characteristics, or variable turbidity of the fluid placed on the surface of the chemically reactive means. Albarella's device does not provide any means to inform the user that the test has failed. For example, transfer of a sample to the reagent pad that contains a low quantity of the reagent of interest (e.g. a low glucose urine sample) and therefore does not activate the reagent pad would produce the same result as a failure to deliver sample to the reagent pad. The reagent area of the Albarella product is directly exposed to the ambient environment. Variation in ambient humidity, for example, introduces an unpredictable amount of sample evaporation and evaporative cooling, both of which can affect sample and reagent reaction kinetics in unpredictable ways, thereby increasing the potential for an incorrect result and inappropriate treatment.

The performance of a diagnostic system is characterized, in part, by its specificity, which is the risk of generating either a false positive (FP) or false negative (FN) result. Depending upon the objective of the assay, one particular failure mode is usually considered a lesser risk. For example, tests for HIV are often designed so that when failure occurs, the failure produces a greater number of false positives since a FP can trigger result verification and at worst unnecessary treatment, whereas a false negative result can withhold treatment leading to further progression and proliferation of the diseases with potentially catastrophic results. Albarella's device provides no means to bias test results, where necessary.

The Albarella device also does not preserve identical spatial relationships between the reagent pad and multiple reference colors. For example, referring to FIG. 14, each reference color is either not in direct contact with the reagent pad or in contact differently. This variation, in and of itself, is known to introduce variation in perceived similarity between the colors of the reagent and reference pads. Another significant problem with the Albarella et al. device is the requirement for the user to directly handle the sample fluid, such as by dropper or pipette. This direct contact creates a high level of biohazard for the user as well as opportunity to alter the composition of the fluid. There is also moderate potential to mis-register the fluid specimen to the test device. Finally, the limited area of the reagent pad and reference color areas makes it very difficult to use this area to contain instructions that can be reliably utilized to instruct the user how to properly obtain and analyze the excrement sample and act on the results.

U.S. Pat. No. 4,980,298 to Blake et al. discloses a device for carrying out chemical or clinical testing of a liquid sample, for example a urine sample, by a specific binding assay, said device compromising a test component which has a sensitized solid surface carrying an immobilized component of a specific binding pair relevant to the assay, and a handling piece, and characterized in that the test component bearing the sensitized surface is removably mounted in spaced relationship with a removably mounted accessory component carrying an accessory solid surface, and in that there is a space between the sensitized surface and the removable accessory component to act as a container for sample liquid, so that when the device is contacted with a sample liquid sources or immersed in liquid which is to provide the test sample, liquid of the sample can enter the space to contact the sensitized surface, and the accessory surface acts to retain and contain sample liquid in contact with the sensitized surface even after removal of the device from further contact with or immersion in the source of sample liquid, and in that the test component is so formed that after removal of the removable accessory component the sensitized surface is left exposed and accessible to further treatment liquid such as washing liquid and/or reagents.

The Blake et al. device is quite complicated and discourages private use in a home setting. The Blake device requires the use of a precisely controlled reaction space to provide, for example, capillary flow to deliver either test sample or subsequent reagents to the chemically activated surface, or to ensure adequate chemical transport of reactants to and from the chemically activated surface during the chemical reaction period, such as is required by bound-free or competitive binding assays. As disclosed, it requires multiple surface reactions (e.g. binding and bound-free separation) and specific binding pairs to analyze the sample. The Blake device requires the use of a separate accessory piece to make possible the multiple chemical reaction steps required to produce a result. The need to alternately attach and remove the separate accessory device to enable the chemical reaction and to interpret the results of the chemical reaction creates additional operating steps and extra disposal. These requirements not only make the device more difficult to use but also exposes the user to an enhanced level of biohazard. It also utilizes a very small chemically activated surface, the absence of a color comparator, and the potential for the optical properties of the sample to adversely affect the developed color of the chemically activated surface as described earlier, makes it very difficult to accurately interpret the results of the chemical reaction.

U.S. Pat. No. 4,943,416 to Kikuchi et al. discloses an automatic urinalysis system which can be readily installed at an excretion site such as a toilet and by which means a subject itself can test its urine easily at any time and can obtain results of such test.” The system described by Kikuchi describes a system that “comprises a sample collecting means for collecting a sample of urine within a stool or the like at an excretion site, a guiding means for introducing the collected urine sample into a testing area within a body of the system, a urine testing element located within the system body, a contacting means for automatically contacting the urine test element with the urine sample in the testing area, a urine testing means for automatically testing the urine test element contacted with the urine sample by the contacting means, a display means for displaying test data from the urine testing means, and a discharging means for discharging the urine sample into the stool after the urine sample has been contacted by the urine testing element.

Kikuchi is complex and requires the use of toilet-sized device, which places severe limitations on device miniaturization, cost, and general availability. The Kikuchi device also requires the active participation and prior knowledge on the part of the end-user, which precludes the device from being used by infants, the incapacitated elderly, or otherwise uncooperative or disabled patients. The completely automated operation described by Kikuchi does not permit user intervention and control, such as to conduct quality assurance checks (e.g. verify adequate sample volume or absence of stool interference).

U.S. Pat. No. 4,789,629 to Baker et al. discloses a method and device for collecting and testing fecal occult blood which permits multiple analyses of a single fecal sample. The slide contains a pocket-like member on a portion of the inside front cover of the slide. An absorbent insert is disposed in the pocket. When the cover is in a closed position the pocket overlies the fecal smear on the specimen viewing sheet and the insert can be removed from the pocket. This design permits an analysis to be done on the specimen receiving sheet of the slide together with a second confirmatory test on the insert.

Baker requires a multiple step testing regime with the direct participation of a medical professional. Because Baker tests a solid, the sample must be actively manipulated and smeared onto the device. Once the sample is manipulated onto the device, a testing reagent must be subsequently applied to the test media to effect a test result, thus requiring a professional to perform the test. Alternatively, a patient or care-giver must be exposed to potentially dangerous or caustic chemicals, with which they have little knowledge.

Accordingly, it is clear that there is a long standing and unmet need for a discrete and self-contained device and method for the non-invasive collection, handling, testing, and/or interpretation of body liquid samples in which all requisite steps may conveniently occur in a single unit, which may be disposable. The body liquid most commonly tested in the non-healthcare environment, such as at home or in an institution, is urine and a particularly strong need exists to passively collect urine from subjects, such as cooperative or non-cooperative young and old patients that minimizes the contamination of the urine and allows for rapid delivery of the urine from the collection site to the testing site. There also is a need for a simple, easy-to-use, reliable and safe device that can provide objective information about a child's hydration status without requiring complicated instrumentation or the diaper of the subject.

### BRIEF SUMMARY OF THE INVENTION

The invention meets one or more of these needs and permits more effective collection and testing of urine from infants and incontinent patients in a home use environment as well as in a healthcare environment, thereby expanding the availability of urine tests for a variety of conditions into a substantially larger population. Thus, the invention greatly enhances the ability to collect and test urine for the assessment of hydration status, and numerous other diseases or conditions, in an individual who may be at risk for dehydration as the result of diarrheal illness and other conditions, or suffering from other health problems. The invention also reduces the potential for improper collection and analysis of the sample, and may be applied to numerous other liquid testing applications as clear from the discussion herein. The invention may be implemented in a number of ways.

According to one aspect of the invention, a non-invasive urine collection and analysis device includes a support body, a collector to retain a urine sample from a subject, the collector including a first engagement surface to releasably hold the collector in a desired location relative to the subject, a urine analysis element including at least one material capable of generating a signal when contacted with urine, the urine analysis element being located in a first position on the support body, the support body including a second engagement surface to hold the collector in a second position spaced from the first position and in proximity to the urine analysis element, and an alignment mechanism to register the collector relative to the urine analysis element and permit transfer of urine from the collector to the urine analysis element.

The collector may include an absorbent, bibulous, or porous medium capable of absorbing urine, and the medium may include at least one of woven and non-woven fabrics, gels, fleece, flock, sponge, and capillary beds. The collector may be constructed of non-irritating and hypo- or non-allergenic, medical grade materials. The collector releases at least a quantity of the collected specimen sufficient to activate the urine analysis element after registration. The collector may imbibe at least about 20 μl of urine from the urine sample and the quantity of collected specimen may be at least about 1 μl. The collector may be non-reactive with at least one of the urine sample and the analysis element. The collector may also include at least one component capable of entering into a first physical, chemical, electrochemical and/or biochemical interaction that modifies the urine sample to facilitate subsequent testing and/or analysis steps. The at least one component may include at least one of (i) antibodies directed against one or more desirable or undesirable constituents of the urine sample to effect separation of the constituents, (ii) chemical buffering agents that cause the urine sample to achieve a particular desired pH or range of pHs, and (iii) capillary flow channels that permit separation of sample components according to their viscosity. The collector should permit only insignificant losses of the urine sample that do not clinically affect the outcome of the analysis of interest. The collector may adhere to a substrate situated in a fixed or substantially fixed position relative to a source of the urine sample, the fixed or substantially fixed position being non-invasive and non-damaging to the substrate and to the source and its immediate surroundings.

The first engagement surface may be configured to releasably affix to a diaper and to the second engagement surface of the support body. The second engagement surface may releasably hold the collector in the support body to facilitate transportation of the collector to another location after use. Alternatively, the second engagement surface may permanently connect the collector to the support body to facilitate disposal after use.

The support body may include instructions that describe graphically and verbally how to engage and release the collector relative to the subject. The support body may be in the form of a foldable booklet. The foldable booklet may includes first and second portions separated by a hinge, with the first position being disposed on one of the portions and the second position being disposed on the other of the portions, and the alignment mechanism may include an outline of the collector on one of the portions. The support body may be formed from non-reactive materials. The support body permits transmission of the signal from the urine analysis element. The signal may be generated due to at least one of a chemical, biochemical, electrochemical or photochemical reaction and may include at least one of a visible color change, change in emission or absorption of visible, ultraviolet or infrared light, change in voltage or impedance, change in resistance, and change in other electrochemical or photochemical property. The alignment mechanism brings the collector and the urine analysis element into direct contact, allowing direct transfer of urine from the collector to the urine analysis element such that no additional structure is required to effect transfer of urine from the collector to the analysis element.

The urine analysis element may include may include at least one reagent having at least one property that is changeable by a reaction between urine and the at least one reagent, and wherein the changed property is perceptible to a user. The at least one reagent may include first and second sides, the first position of the support body enabling urine to be applied solely to the first side of the at least one reagent and the second side to be viewed by a user, and wherein urine is applied to the side of the at least one reagent that is not viewed by the user. The analysis element may include a filter to remove undesirable chemical and biochemical interference in the urine sample and prevent undesirable properties of the urine sample from influencing the operation of the analysis element. The at least one material of the urine analysis element may include a single reagent, or multiple reagents, each testing for a different characteristic of the urine sample. The analysis element may be maintained in sealed, humidity controlled environment in the booklet to minimize biohazard risks after use.

The system may further include an evaluation device to provide a result based upon the signal or a characteristic of the urine analysis element. The evaluation device may include a color reference chart printed directly on the support body. Alternatively, the evaluation device may include at least one of biosensors, photo sensors, electrochemical sensors, circuit elements, and signal processors, and an output capable of providing a user with a result. The result may include at least one of binary information or linear information in qualitative, semi-quantitative, or quantitative format wherein the evaluation of the result may be performed by a user without specific training. The evaluation device may provide specific instructions about how to proceed following interpretation of the result. Thus, the user may be instructed to establish contact with a healthcare provider immediately upon receipt of a specific result. The evaluation device may provides a user with technical information that the user may transmit to a person with a higher level of training, thereby providing quantitative or semi-quantitative information to the more highly-trained person. The technical information may include a value for at least one of urine specific gravity, glucose, protein, bilirubin, urobilinogen, red blood cell, white blood cells, nitrites, uric acid, creatinine, pH, leukocyte presence, nitrites, ketones, human chorionic gonadotropin, pharmaceuticals, organic acids, sexually transmitted diseases, metabolic products of oxidant damage to tissue, genetic material in the form of DNA and/or RNA specific to the host and particular organisms, antigens from bacteria, and antigens from parasites. The evaluation device also may indicate the failure of the analysis system to produce the result (“No Result”). In particular, the urine analysis element may include a colored reagent strip in which the color of the reagent strip prior to liquid activation is included in the reference color scale thereby providing an indication of the “No Result” condition. The evaluation device may include a color reference chart having multiple colors, the chart being disposed so that each reference color is situated equidistant from the urine analysis element. The evaluation device may include a color reference chart having multiple color zones of unequal size, which maximize the likelihood that, when in doubt, a user chooses the most conservative result, thereby increasing the test's probability of producing the safer of two alternatives, either an increased false-positive or false negative rate. The color zones may be generally biased to correct for variations in the results produced by a variable property of the sample, such as pH. The evaluation device may not contact the urine sample. The evaluation device may include a color reference chart that is brought into proximity with but does not contact the analysis element. The support body may include a transparent window and the color reference chart is disposed on the opposite side of a transparent window that isolates the analysis element.

According to another aspect of the invention, a non-invasive specimen collection and analysis device includes a support body, a collector to retain a specimen from a subject, the collector including a first engagement surface to releasably hold the collector in a desired location relative to the subject, an analysis element including at one least material capable of generating a signal when contacted with the specimen, the analysis element being located in a first position on the support body, and a transfer mechanism to selectively permit transfer of the specimen from the collector to the first analysis element.

The specimen may be a biological sample, such as urine, saliva, blood, sweat, tears, plasma, serum, milk, spinal fluid, lymph fluid, secretions from the respiratory tract, secretions from the intestinal tract, and secretions from the genitourinary tract. In particular, the body liquid may be urine in which case the source of the liquid is the urethra of an infant or other person wearing a diaper or incontinent device. In this case, the first engagement surface may include adhesive allowing the collector to be releasably affixed to the diaper or incontinent device in a substantially fixed relationship to the urethra.

The collector may include at least one component capable of entering into a first physical, chemical, electrochemical and/or biochemical interaction that modifies the specimen to facilitate subsequent testing and/or analysis steps. The at least one component may include at least one of (i) antibodies directed against one or more desirable or undesirable constituents of the specimen to effect separation of the constituents, (ii) chemical buffering agents that cause the specimen to achieve a particular desired pH or range of pHs, and (iii) capillary flow channels that permit separation of specimen components according to their viscosity.

The support body may include a plastic holder, such as a booklet, supporting the analysis element. The support body may include an orifice in communication with the collector.

When the specimen is urine, the analysis element may include at least one reagent capable of producing the signal in response to contact with urine, the signal containing information about characteristics of the urine. The signal may contain information about at least one urine specific gravity, urine nitrite content, and urine leukocyte esterase content. The signal may also contains information about at least one or more of glucose, protein, bilirubin, urobilinogen, red blood cells, white blood cells, nitrites, uric acid, creatinine, pH, ketones, human chorionic gonadotropin, pharmaceuticals, organic acids, sexually transmitted diseases, genetic material in the form of DNA and/or RNA specific to a particular organism, metabolic products of oxidant damage to tissue, antigens from bacteria, and antigens from parasites.

When the specimen is saliva, the analysis element may include at least one reagent capable of producing the signal in response to contact with saliva, the signal containing information about characteristics of the saliva. The signal may contain information about at least one of hydration, therapeutic drugs, such as theophylline, and endogenous steroids, such as cortisol.

The device may include an alignment mechanism to register the collector and the analysis element prior to transfer of the specimen to the analysis element. The alignment mechanism may include an outline of the collector on the support body. The alignment mechanism may also include a hinge operable to bring the collector into direct contact the analysis element. Alternatively, when the source of the specimen is the urethra of a continent person, the alignment mechanism may include a gripping portion of the support body to facilitate manually placing the collector in a stream of liquid.

The transfer mechanism may include a separator sealingly disposed between the collector and the analysis element. The separator may include an impermeable sheet connected to the support body via a frangible connection. The separator may movably interact with the support body. The separator may take the form of one or more of an ampule, blister packet, capsule, compartment, container, and balloon.

The device may further include an evaluation device to provide a result based upon the signal or a characteristic of the analysis element. The evaluation device may include a color reference chart printed on the support body and disposed in a fashion to surround a “window” through which the analysis element is visible to a user. The analysis element may include first and second analysis elements each including at least one material capable of generating different reactions when contacted with the specimen.

The device also may include an optional distribution element to spread the specimen over the analysis element. The support body may include a liner impermeable to liquid, the liner supporting the collector, the analysis element and the transfer mechanism. The transfer mechanism may include an impermeable shield.

According to another aspect of the invention, a method for testing liquids obtained non-invasively from an animal or human subject includes the steps of positioning a collecting device in a desired location relative to the subject, collecting a liquid sample from the subject in the collecting device while it is in its desired location relative to the subject, supporting the collecting device in a fixed special relationship to an analysis element, registering the collecting device with the analysis element, and transferring liquid from the collecting device to the analysis element to enable a reaction to occur between the liquid and the analysis element, with the reaction being indicative of a characteristic of the liquid.

The method may also include the step of evaluating the results of the analysis based upon the characteristic to indicate a condition of the subject.

The method may also further include the step for transporting the collecting device to a location in proximity to the analysis element. The transporting step may be accomplished by a user without specific training, permits preservation of the integrity of the sample, and obviates the need for any further processing prior to being transported to a location in proximity to the analysis element.

The registering step may include the use of instructions included with the support body that describe graphically and verbally how to properly register the collection device and the analysis element such that proper registration is evident to a user without specific training.

The supporting step should not cause premature interaction of liquid in the collecting device and the analysis element. The supporting step may include permanently attaching the collection device to a support body including the analysis element to facilitate subsequent easy disposal of the collection device and analysis element. Alternatively, the supporting step may include removably attaching the collection device to a support body including the analysis element such that the collection device may be removed from the support body following the transferring step, thereby allowing the collection device to be safely transported to another location without the support body.

The transferring step may include one of i) directly contacting the analysis element with the collecting device, ii) removing a separator between the analysis element and the collection device, and iii) breaking an ampule.

The method may further include the step of preparing the liquid sample in the collecting device for analysis. The preparing step may include releasing genetic material from the sample.

The analysis element may not contact the subject in any of the steps to reduce risk of an adverse reaction between the materials used in the analysis element and the subject. The steps may occur in a support body providing a user with control over the timing and location of each of the steps in the testing process. The method may further include introducing at least one discrete and variable time interval between the transporting and registration steps such that activation of the analysis element occurs without substantial loss of function of the test or its validity.

According to a further aspect of the invention, a non-invasive urine collection and analysis device includes means for supporting the device, means for collecting a urine sample from a subject, the collecting means including means for releasably holding the collecting means in a desired location relative to the subject, means for analyzing the urine sample, the analyzing means being located in a first position on the supporting means, and means for aligning the collecting means relative to the analyzing means to permit transfer of urine from the collecting means to the analyzing means. The device may further include means for evaluating the result of the analyzing means.

According to yet a further aspect of the invention, a collection device for a biological sample obtained from a subject includes a medium capable of absorbing and retaining a biological sample directly from a subject and selectively releasing at least some of the sample to an analysis element in response to an external force, and an engagement surface to hold the medium in a plurality of desired locations relative to the subject and the analysis element.

The engagement surface may hold the medium in the desired location relative to the subject via a releasable connection, and may hold the medium in the desired location relative to the analysis element via a permanent connection. If the biological sample is urine, the engagement surface may releasably hold the medium in a desired location in a diaper worn by the subject, and may hold the medium in a desired location in a support body containing the analysis element. The medium may release at least some of the biological sample in response to an external force applied to the medium via the support body. The external force may include at least one of a compressive force and gravity.

The medium may include a pad made of an absorbent, bibulous, or porous medium capable of absorbing urine. The medium may include at least one of woven and non-woven fabrics, gels, fleece, flock, sponge, and capillary beds. The medium may be constructed of non-irritating and hypo- or non-allergenic, medical grade materials, and be non-reactive with at least one of the sample and the analysis element. The medium may imbibe at least about 20 μl of the sample and release at least about 1 μl after application of the external force. The medium may also include at least one component capable of entering into a first physical, chemical, electrochemical and/or biochemical interaction that modifies the sample to facilitate subsequent testing and/or analysis steps. The at least one component may include at least one of (i) antibodies directed against one or more desirable or undesirable constituents of the sample to effect separation of the constituents, (ii) chemical buffering agents that cause the sample to achieve a particular desired pH or range of pHs, and (iii) capillary flow channels that permit separation of sample components according to their viscosity. The medium permits only insignificant losses of the sample that do not clinically affect the outcome of the analysis of interest. The medium may adhere to a substrate situated in a fixed or substantially fixed position relative to a source of the sample, with the fixed or substantially fixed position being non-invasive and non-damaging to the substrate and to the source and its immediate surroundings.

The engagement surface may be configured to releasably affix to a diaper. The device may be used in combination with a support body containing the analysis element.

According to yet another aspect of the invention, a process for the analysis of very small volumes of a biological liquid includes the steps of: collecting a first volume of biological liquid from its source without the use of instrumentation and without the direct participation, manipulation, or modification of the source; directly transferring a minimum quantity of a second volume from the collected volume sufficient to an analysis system to activate the analysis system at a location remote from the source without intervening manipulation of the second volume; and producing a signal containing information about at least one property of the biological liquid.

The process may further include the step of evaluating the signal. The evaluating step may include evaluating the signal by a user not specifically trained in the analysis being conducted.

The liquid being tested may be a human body liquid and the first volume may be at least about 20 μl, and the second volume may be at least about 1 μl. The human body liquid may be one of urine, saliva, blood, sweat, tears, plasma, serum, milk, spinal fluid, lymph fluid, secretions from the respiratory tract, secretions from the intestinal tract, and secretions from the genitourinary tract.

The collecting step may include isolating the first volume from the analysis system. The transferring step may include contacting the second volume with at least one chemically, biologically, biochemically, or electrochemically reactive analysis element after a time period selected by the user expires.

The invention has a number of features and advantages over the prior art. For example, with respect to sample handling, the prior art generally requires the user to manually transfer and precisely register the sample liquid to the analysis component. Embodiments of the invention may greatly simplify this step and provide an inherently high level of certainty that sample liquids will be properly transferred to the analysis component. This may be accomplished by utilizing a sample collection pad area that is significantly larger than the reagent area, and the use of a test booklet and printed references for properly positioning the sample collection pad against the reagent pad, all of which significantly reduce user-related errors.

With respect to control of reaction conditions, the prior art typically allows the reagent areas to be directly exposed to the ambient environment, where variation in ambient humidity, for example, introduces an unpredictable amount of sample evaporation and evaporative cooling, both of which can affect reagent reaction kinetics, thereby influencing the result in unpredictable ways. Embodiments of the invention may provide a sealed and more controlled environment to ensure, for example, that sample evaporation and evaporative cooling are minimized and held constant.

With respect to control of detection specificity, the performance of a diagnostic system is characterized, in part, by its specificity, which is the risk of generating either a false positive (FP) or false negative (FN) result. Depending upon the objective of the assay, one particular failure mode is usually considered a lesser risk. For example, tests for HIV are often designed so that when failure occurs, the failure produces a greater number of false positives, since a FP can trigger result verification and at worst unnecessary treatment, whereas a false negative result can withhold treatment leading to further progression and proliferation of the diseases with potentially catastrophic results. In one particular embodiment of the invention in which urine is tested for specific gravity so as to infer the hydration status of the subject, the assay is designed to favor a false positive (FP) result in the event of a test failure, since this outcome produces an unnecessary intervention (e.g. administration of liquids), as opposed to a false negative result which could result in withholding liquids and exacerbating the condition of a truly dehydrated patient. Thus, embodiments of the invention may provide for active control of detection specificity.

With respect to the breadth of the results, embodiments of the invention may provide for a greater range of results than that of the prior art, such as Albarella, through the use of e.g., multiple reference colors, as well as a No Result Condition, as discussed in more detail subsequently.

With respect to chemical, biochemical and optical interferences, the prior art typically requires visual interpretation of the same surfaces to which the sample liquid or excrement is applied, which provides: the potential for chemical or biochemical materials in the sample to interfere with the chemical, biochemical, or electrochemical reactions taking place within the reagent strip a chip-based detector; or for optical properties of the sample, such as sample reflection, refractive index, or color or turbidity, to interfere with the interpretation of the activated reagent strip, thereby producing an incorrect result. Embodiments of the invention help to avoid these problems by using a reagent strip that may be read through the non-wetted side, versus the wetted side as in the prior art. This aspect of the invention creates a filtering effect that helps to neutralize undesirable optical properties of the sample as well as undesirable chemical and biochemical interferences. By viewing the test result through the back or non-wetted side of the reagent strip, one is more likely to view the intended results of the chemical reaction. Embodiments of the invention may also maintain a controlled and constant optical path between the reagent and the eye. This feature may reduce or eliminate variation in test result interpretation due to such factors as variation in sample refraction, sample induced variation in surface reflection, or sample turbidity. Prior art devices can expose the sample chemical compounds in the absorbent article (e.g. diaper) known to affect, for example, urine ionic strength. Embodiments of the invention may avoid this by isolating the collection device from the absorbent article.

With respect to sample interferences with reference colors, sample liquid and excrement have optical features, such as color and opacity, as well as the ability to alter the reflectance and refractive properties of any surface it is in contact with. These properties can inappropriately alter the appearance of a reference color and lead to inaccurate interpretation of a chemical reagent strip or other analysis component. Unlike prior art devices, embodiments of the invention may avoid this problem by not allowing the sample liquid to contact the reference colors.

With respect to reference colors, most prior art devices place reference colors on a completely separate component (usually the bottle or other packaging of the test strips); the Albarella patent cited above teaches fixed placement of the reference colors adjacent to the test strip. Both methods have inherent disadvantages; in the former, the user must move the test strip back and forth to find the best color match, while in the latter the reference color chart is subject to the errors described above. Embodiments of the invention may provide for elimination or reduction of both kinds of disadvantages, providing for greater latitude in the selection of the reference colors as well as control of their integrity (i.e. the reference colors may not come into contact with the sample liquid or excrement.) By placing the reference colors on a separate component of the device from where the reagent is located while maintaining the components in fixed spatial relationships, the user gains control over the manipulation of the reagent chart without sacrificing the precision offered by reliable registration of the reagent and the color chart. Reference colors may be brought into proximity to the reagent pad, for example, by using the hinge feature of the booklet.

With respect to interpretation of test results, embodiments of the invention may utilize reference colors that are oriented circularly around the reagent pad, thereby preserving the spatial relationship identically for each reference color with respect to the reagent pad. In addition, the size of the reference color blocks increase with rising specific gravity indications. These features can be important, since it has been shown that variation in spatial relationship or size alone, between objects of comparison, can affect the interpretation of relationships between the objects. Both features provide increased reliability and again predispose the reader towards the more concerning “high” indication, tending to produce the desired false-positive mode in the event of a misinterpretation.

With respect to user instructions, embodiments of the invention may utilize integrated instructions in multiple locations to guide the user in performing the test and reporting the result to the healthcare provider. Guidance is specifically provided to help obtain the specimen, transport the specimen to the test site, performing the test, reading the result, interpreting the result, reporting the result, and acting on the result. This, along with some of the other advantages discussed above, reduces the chance of reading errors and/or incorrect treatment.

With respect to feedback as to whether the test device functioned properly, prior art devices typically require the user to make multiple, unguided observations of the testing system to deduce whether or not the test performed properly, making it possible that a device failure could go undetected and lead to an incorrect diagnosis and treatment. Embodiments of the invention may provide positive feedback to the user whenever the testing system fails to perform properly to ensure that any such failure does not lead to an incorrect diagnosis and treatment. One way this is done is to preserve the color of an inactivated reagent strip while providing a similar reference color labeled as a “No Result” condition.

Embodiments of the invention may also increase safety to the wearer of the disposable absorbent product by eliminating chemical contact with the skin, and/or by containing and enclosing the sample liquid and/or excrement thereby greatly minimizing any chance for contact with the user. This feature also may simplify disposal of the biological sample.

The invention may also provide a much simpler assay design than the prior art. For example, the above-discussed patent to Blake, et al. requires the use of a precisely controlled reaction space to provide, for example; capillary flow to deliver either test sample or subsequent reagents to the chemically activated surface; or to ensure adequate chemical transport of reactants to and from the chemically activated surface during the chemical reaction period, such as is required by bound-free or competitive binding assays. Embodiments of the invention may provide for a much simpler assay design, which do not require a precisely controlled reaction space, since one-time application of finger pressure is sufficient to properly transfer the sample to the reagent pad; thus, device operation maybe much less sensitive to user variability. Embodiments of the invention may also allow a single step reaction to take place on or within the reaction pad. The Blake device utilizes multi-step surface reactions and specific binding pairs, which is disadvantageous because the increased number of steps and binding specificity create opportunities for functional error and incorrect results. The Blake device also utilizes a solid accessory device which adds to the complexity of their assay: Fluid is held in liquid form (versus the absorbent pad of the invention) which creates additional liquid handling requirements, risk of spillage, and enhanced biohazard risk. The accessory device must also be removed to enable subsequent assay steps, including reading. Reading is further compromised due to the lack of a comparator adjacent to the reagent area.

Some of the prior art, such as Kikuchi, et al. and Baker et al., require the active participation and prior knowledge on the part of the subject, which is not required in embodiments of the invention. Indeed a system like Kikuchi, et al. could not be used with infants, debilitated geriatric patients, or other patients who cannot cooperate in the process. Kikuchi, et al. also requires the use of very complex instrumentation, which increases design and use complexity.

Everhart, et al. describe the use of a device that is integrated into a functional disposable unit (e.g. diaper), which is disadvantageous because it may place reactive chemicals in close proximity to a patient's skin and may cause an unwanted reaction. In contrast, embodiments of the invention may use a sample collection pad that is inserted and removed for chemical reaction at a remote site.

Accordingly, the use of one or more features of the inventions generally creates a superior testing method as discussed above. Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 is a perspective view of a first embodiment of a booklet device for collecting and testing urine samples constructed according to the principles of the invention illustrating a sample collection pad and a test strip selectively isolated from the sample collection pad.

FIG. 2 is a plan view of the booklet in FIG. 1 illustrating the outside cover of the device with two reagent windows.

FIG. 3 is a perspective view of the booklet in FIG. 1 illustrating the outside of the device in a closed position.

FIG. 4 is a perspective view of the booklet of FIG. 1 illustrating a modification having a transparent outer cover and the juxtaposition of the sample collection pad with the reagent test strip.

FIG. 5 is a plan view of the booklet in FIG. 1 illustrating a modified inside cover having a single reagent test strip.

FIG. 6 is a plan view of a sample collection pad constructed according to the principles of the invention, which may be used with the embodiments of FIGS. 1-5.

FIG. 7 is a cross sectional view of a typical sample collection pad shown in FIG. 6 showing the various layers from which it may be formed.

FIG. 8A is a perspective view of a reagent test strip constructed according to the principles of the invention having two reagent pads.

FIG. 8B is a perspective view of a reagent test strip constructed according to the principles of the invention having two reagent pads and an absorbent pad located between the reagent pads.

FIG. 9 is a schematic illustration of the instructions and packaging constructed according to the principles of the invention which may be used in any of the embodiments in FIGS. 1-9. This figure also illustrates the circular arrangement of the reference color panel around the reagent test area.

FIG. 10 is a top plan view of a second embodiment constructed according to the principles of the invention having a pull tab for selectively isolating the sample collection pad and test strip.

FIG. 11A is a side plan view of the device of FIG. 10.

FIG. 11B is a perspective view of the device of FIG. 10.

FIG. 12 is a side, perspective exploded view of a third embodiment of the invention having a pull tab for selectively isolating the sample collection pad and test strip, and a channel lock system.

FIG. 13 is a side plan view of the device of FIG. 12.

FIG. 14 is an illustration of a calorimetric scale of the invention that may be juxtaposed with a reagent test strip of the invention.

FIG. 15 is a top plan view of a fourth embodiment constructed according to the principles of the invention adapted for use for continent users having a pull tab for selectively isolating the sample collection pad and test strip.

FIG. 16 is an exploded illustration of the device of FIG. 15 showing the arrangement of the major constituents.

FIG. 17 is a side plan view of a fifth embodiment constructed according to the principles of the invention having an ampule for selectively isolating the reagent from the sample collection pad.

FIG. 18 is a top perspective illustration of the device of FIG. 17 showing the ampule in the middle of the device.

FIG. 19 is a top plan illustration of a sixth embodiment constructed according to the principles of the invention having a sample collection pad and a test strip integrated into the sample collection pad.

FIG. 20 is a schematic illustration of the instructions for interpreting a dual reagent test pad, wherein the results of one pad are used to interpret the results of a second pad, constructed according to the principles of the invention and which may be used in any of the embodiments in FIGS. 1-19.

### DETAILED DESCRIPTION

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scales, and features of one embodiment may be employed with other embodiments as the skilled artisan would recognize, even if not explicitly stated herein. Descriptions of well-known components and processing techniques may be omitted so as to not unnecessarily obscure the embodiments of the invention.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It is also noted that as used herein and in the appended claims, the singular forms “a,” “an,” and “the” include the plural reference to one or more reagents, testing areas, and equivalents thereof known to those skilled in the art.

Unless defined otherwise, all technical and scientific terms used herein have the same meanings as commonly understood by one of ordinary skill in the art to which the invention pertains.

The examples used herein are intended to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings.

Provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

A “sample” refers to a sample of tissue or liquid from a human or animal including, but not limited to urine, plasma, serum, spinal fluid, lymph fluid, DNA, RNA, or other biological materials in such liquids, the external secretions of the skin, secretions from the respiratory, intestinal and genitourinary tracts, tears, saliva, milk, blood cells, tumors, organs, tissue and sample of in vitro cell culture constituents.

“Patient,” as used herein, includes individuals who require or may require intervention or manipulation due to a known or suspected disease state, disease predisposition, treatment regimen or experimental design. Furthermore, the term “subject” includes animals and humans. Thus, when referring to processes such as collecting a sample from an animal, it is intended that the animal can be a human. Although at times reference may be made herein to “an animal or human,” this is not intended to imply that the term “animal” does not include a human.

“Reagent,” as used herein, includes any substance used to detect, measure, examine, or produce other substances, and known to react in a specific way. A “reagent” can include a test substance that is added to a system in order to bring about a reaction or to see whether a reaction has occurred. “Reagent” may be used interchangeably with “reactant.” As used herein, “reagent” can include chemical, biological, radiological, or electrical means.

The embodiments of the invention relate to methods and devices for chemical, biochemical, electrochemical, and/or clinical testing and their use. The embodiments include improved apparatus for carrying out such tests, and provide specific improvements in making such tests available for use in situations and populations in which such testing has heretofore been impractical, uncomfortable, and/or dangerous. In general, the embodiments disclosed herein provide methods and devices for the non-invasive collection of a liquid sample in small quantities, directly at the source of that liquid sample, isolation of the collected sample from a testing site, the transportation of that sample to a site for testing contained within the device itself, and the selective application of some or all of the specimen to a testing apparatus contained within the device itself, with a minimum of specimen handling required. Other aspects include a method and device for facilitating the reading of the result of the specimen test by a skilled or unskilled user, including means for determining that no adequate sample has been delivered to the testing site. Embodiments also may allow for sample preparation prior to testing the sample particularly with respect to genetic material. Furthermore, the disclosed methods and devices may facilitate the reporting of the test result by a skilled or unskilled user, and provide for the immediate interpretation of the test result by the user alone or in consultation with a more-highly skilled individual in the field pertaining to the test. Finally, embodiments of the invention provide a method for indicating a correct action to be taken based on each specific result obtainable by the test.

The principles disclosed herein, in their simplest description, allow for the collection of a small volume of a liquid to be tested, isolation of the volume of liquid, transfer of the liquid to a testing area, and then receiving and interpreting the signal produced as the result of the contact between sample and test material. In its simplest embodiment a device constructed according to the principles of the invention may include a minimum of two components: a sample collection component as described below and the body of the device.

While the principles of the invention are particularly suited for use with testing of urine from infants and other incontinent patients, a skilled artisan will realize the general methods and devices disclosed herein may be used in the collection, handling, and testing of a variety of samples and body liquids of various human and animal subjects. Non-limiting examples include collection and testing of urine, saliva, sweat, skin oils, milk, and tears. In a specific non-limiting example of urine testing, determination of urine specific gravity by means of a calorimetric ionic strength indicator is described. In one embodiment, the device may be placed into a diaper or within clothing to be disposed adjacent to the urinary meatus, from which urine emerges. Urine is absorbed into the collection and handling apparatus and upon activation by removal of the impermeable tab, is ultimately directed to one or more calorimetric ionic strength indicators affixed to the transparent base of the device. After the prescribed time interval, the color of the indicators is read by the user and compared with the color chart printed on the device. Interpretation is facilitated by an indicator disposed so as to demarcate “acceptable” from “concerning” values, in a non-limiting example.

Embodiments of the invention also allow one to determine information on a child's hydration status while in the home environment. These embodiments can be used with any type of diaper, pants, underwear, panty, or other support which can be desired. If the urine can reach the device, then the device can be utilized. The invention can employ solid phase chemical testing of the test liquid.

The principles of the invention are not limited by patient species and can be utilized with humans of any age, as well as any mammal, or other animal species whose urine can be contacted with the device. As non-limiting examples dogs, cats, monkeys and pigs can be tested with this invention, with appropriate design modifications as would be apparent to a skilled artisan.

The device is not limited to urine testing, as noted above, but can be utilized with many bodily liquids placed in contact with the device. The invention does not place the subject, or patient, at risk and is non-invasive in that no instrumentation, such as catheters, needles, or other medical devices are used to obtain the liquid sample. The invention reduces risk of harm to the caregiver.

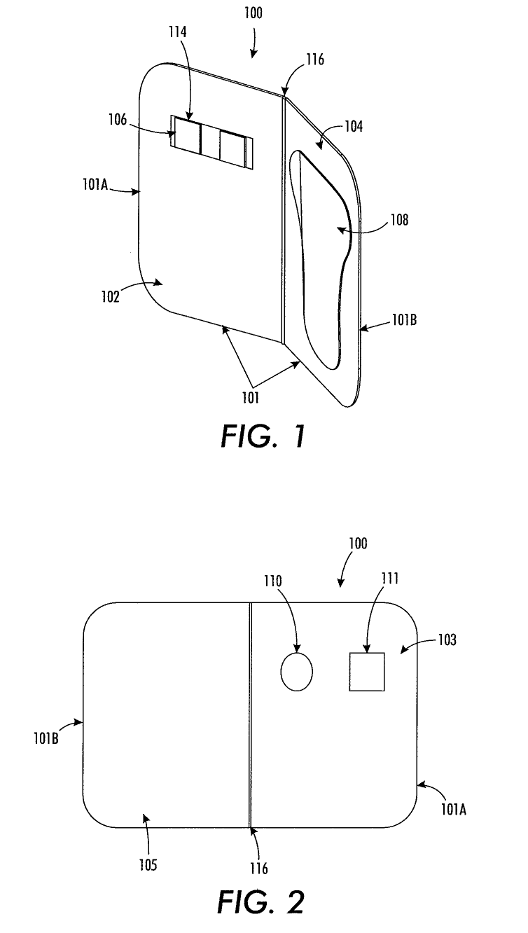

The invention may be embodied in a swabbing or absorbent device, such as shown in FIGS. 1-7 described in detail below, for bringing sample fluid, typically a liquid into contact with a material (non-limiting example of a cloth, diaper, polymer, sponge, cotton or gel). The swabbing or absorbing mechanism of the device contacts and collects a liquid and upon activation the device brings the absorbed liquid into contact with the testing medium.

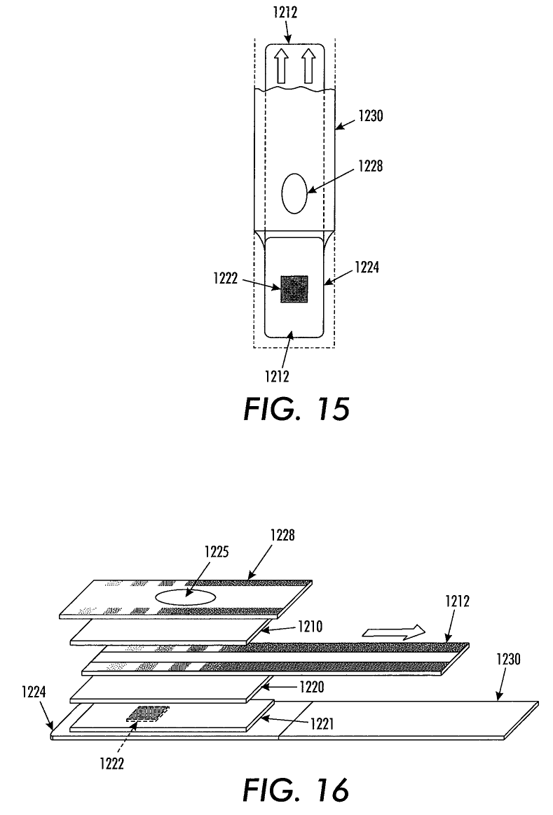

The invention may be embodied in the form of a wand or other bodily liquid collecting device, such as shown in FIGS. 15-16, described in detail below. The testing medium (non-limiting e.g., a test strip) can be embedded, included or in contact with any collecting medium.

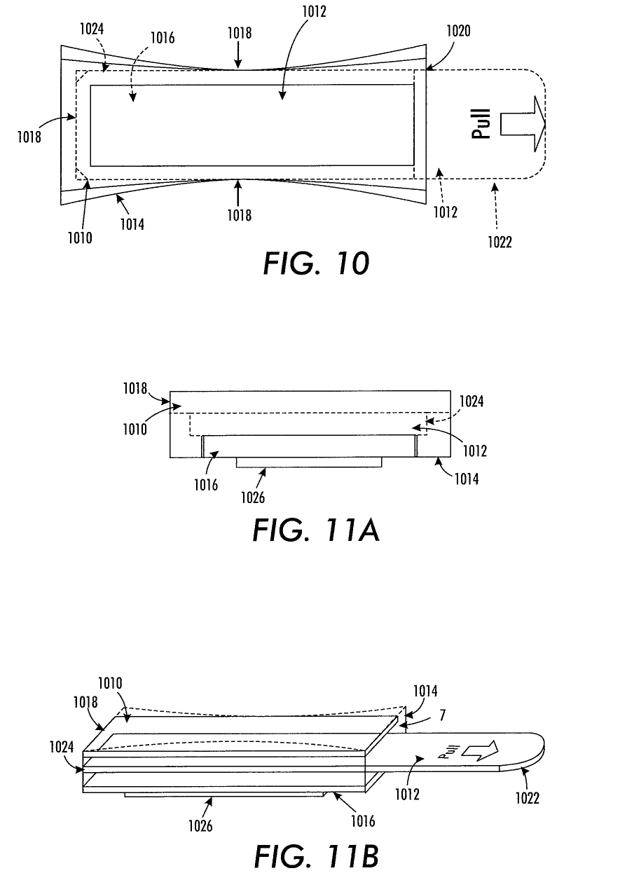

The invention may be embodied so that the testing medium may be separated by a means for isolating the liquid to be collected, such as a pull strip, such as shown in FIGS. 10-13, 15-18, described in detail below. Operation of the isolating means allows the liquid and testing medium to come in contact and for the test medium to provide an indication. In a number of embodiments, the invention allows for the separation of the liquid sample from the test medium until a time the person conducting the test chooses.

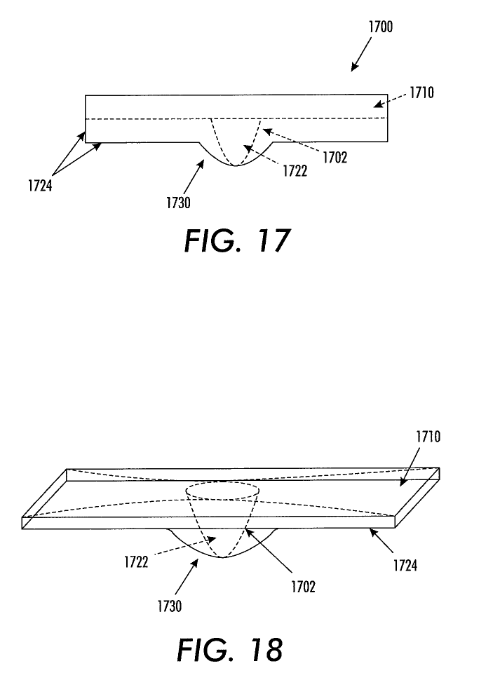

The invention may be embodied so that the testing means is separated in a blister packet, capsule, sealed packet, ampule, compartment, container, balloon or other appropriate device, such as shown in FIGS. 17-18, described in more detail below. The selected testing means can be contained in any appropriate material for a given testing medium including plastic, glass, rubber, polymer, cloth, paper product, or other material.

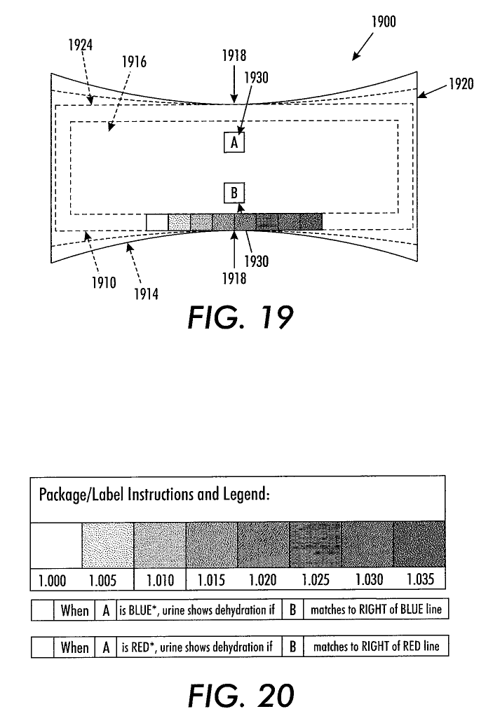

Some of the principles of the invention also may be embodied in a diaper dehydration pad, such as shown in FIG. 19, described in detail below, which can be inserted into a diaper, or be integral with the diaper. The diaper dehydration pad can be worn between the diaper and an infant's body. The invention may be independent of any clothing or diaper support.

A number of embodiments are particularly suited for use as an at-home test for monitoring the hydration of infants. In these embodiments, the device may measure specific gravity (SG) from the infant's urine. The test strip turns a certain color based on SG, providing one indication of whether the infant's hydration status is good, fair, poor, or other desired indication.

A number of embodiments involve collection of a body liquid such as urine by a pad near the wearer's skin, the reception of the urine by a pad near the chemical testing region (separated from the collection area by an isolating means, such as an impermeable shield, until ready for use), and then the distribution of the urine onto the chemical testing region to prevent spreading of test chemical throughout the unit. Where the term “leaching” is used, it as appropriate refers to leaching within the device or unit; the device of these embodiments can prevent leaching of chemical onto the wearer's skin.

The invention may also include a sample preparation step, which is particularly useful when testing for genetic material. In one embodiment of the invention, a sample collection pad of the invention may include at least one element that prepares a specimen to facilitate detection and analysis of genetic material. Such preparation may include, by way of non-limiting example, exposure to biological, chemical, electrochemical, or other means of breaking (lysing) cells to release their genetic material. Biological specimens may contain important analytical and diagnostic information comprised of genetic material (DNA and/or RNA) from both the host and any possible infecting agents (pathogens). Segments of genetic material may be available as short sequences free in a specimen, or they may be present within cellular components of the specimen. Similarly, the collection pad might be pre-treated with antibodies directed against specific desired or undesired components of a specimen so as to effect separation of said components from the balance of the specimen. The collection pad also may include chemical buffering agents, which may affect a sample to achieve a particular characteristic before testing, i.e. a specific pH.

Turning now to the drawing figures, FIGS. 1-5 show embodiments of the invention as a booklet-type device for the collection of urine samples from individuals who are incapable of cooperating with the collection of the sample, for example, an infant in diapers or an incontinent adult. A further feature of these embodiments is a means for transferring the collected urine to a different portion of the device, such means minimizing user contact with the specimen, and the delivery of the urine specimen to a test area contained within the body of the device. In one embodiment the test area includes a chemical reagent-treated test strip. In a further embodiment the reagent-treated test strip is sensitive to urine ionic strength, so as to provide a calorimetric indication that serves as an estimate of urine specific gravity, as is known to those skilled in the art. In one embodiment a means is provided for viewing the signal produced by the test area through an optically-clear protective material situated in a position directly in registration with the test strip, which itself is situated in registration with the wetted sample collection pad, the two assemblies being brought into direct physical contact to initiate the test. Advantages of these features will be evident to one skilled in the art, and include collection of the liquid specimen without active participation of the specimen donor, the ability to initiate the test at the user's convenience, and ready disposability of the unit after the test is completed with minimal contact with the liquid. Other advantages will become clear from the description herein.

Thus, as shown in FIGS. 1-5, the device 100 may take the form of a “booklet” 100. The booklet 100 includes a body 101 supporting a sample collection component 108. The body 101 may include two opposed pages 101A, 101B each having inner and outer surfaces, 102, 103 and 104, 105 respectively. The sample collection component 108 may be supported on the inner surface 104 of page 101B, fixedly or removably by any means known in the art as discussed in more detail below. A testing medium, such as a reagent strip 106, may be supported in a test area 114 provided on the inner surface 102 of page 101A. The body 101 has a midline crease 116, which separates the sample page 104 and the test page 102. The body 101 can be folded along the midline crease 116 to form a living hinge for selectively bringing the component 108 into contact with reagent test strip 106.

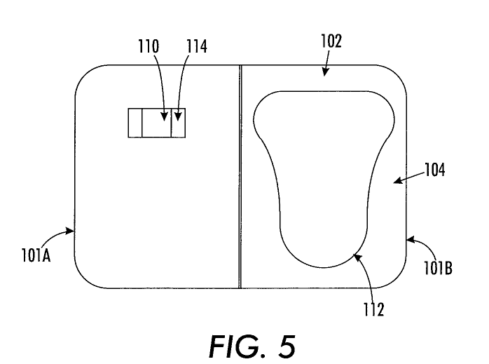

The body 101 of the device 101 may be a thin, semi-rigid booklet formed from one or more clear plastic materials, such as a polymer. As shown best in FIG. 2, outer surface 103 of page 101A may include two clear windows 110, 111, which may be formed in similar or dissimilar shapes, such as oval and square as shown. The test area 114 is situated to overlay the clear windows 110, 111 through which the user may receive a signal indicative of the test results, as described below. In FIGS. 1-4, the booklet contains two windows 110, 111, which correspond to a reagent test strip 106 with two test pad areas; however, as illustrated in FIG. 5, the booklet may have a single window 110 corresponding to a reagent test strip 106 with a single test pad. As discussed below, one side of the window is open to receive the sample liquid and the other may be hermetically sealed by any means known in the art such as a transparent partition. The reagent test strip 106 may be a chemical, electrical, or biological sensor that responds to the characteristic(s) of interest in the tested liquid by producing a perceptible signal. The sensor includes means for transmitting the signal produced by the test to a user, such as the optically-clear windows.

As shown in FIG. 5, the inside surface 104 of page 101B includes a site 112 for placement of the sample collection component 108. The site 112 may be recessed or include an outer peripheral outline to assist the user in proper placement and registration of the sample collection component 108. A transparent page 101A′ is shown in FIG. 4 to demonstrate that the component 108 is positioned so that it is aligned and registered with the test strip 106 when the hinge 116 is operated to close the booklet.

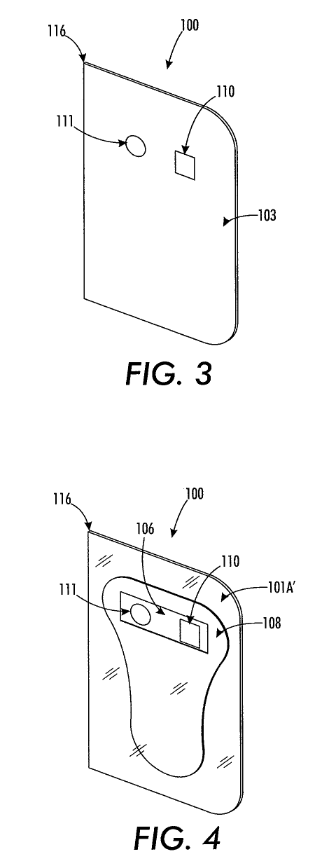

Accordingly, once the sample collection component 108 has been placed into its designated position 112 in the body 101 of the device, the device may be activated by bringing the component 108 into contact with the testing strip 106, as shown in FIGS. 3 and 4. Body 101 supports the pages 101A, 101B in a position such that when component 108 is disposed within site 112, it is substantially in alignment with the reagent strip 106 when the user closes the booklet, bringing the component 108 into direct physical contact with the strip 106. The user may press the component 108 into the strip 106, forcing the collected sample to contact the strip 106. The sample interacts with the strip 106 and undergoes a chemical or biochemical reaction as is known in the art. The now activated strip 106 produces a signal (for example, a color change) that is visible through one or more optically-clear openings 110, 111. FIG. 3 illustrates the booklet embodiment in its activated, closed position folded along midline crease 116. FIG. 4 illustrates the booklet embodiment with a schematic transparent page 101A′. This schematic illustrates the relative positions and subsequent alignment of the sample collection component 108 and the reagent test strip 106 when the booklet has been closed.

Instead of forcing the sample from the component 108, other techniques may be used to contact the sample with an analysis device. For example, the sample collection component 108 may be placed into its designated position within site 112 and the device may be activated by bringing electrically conductive probes into contact with the wetted sample collection component to perform electro-analytical measurements of the sample, such as sample resistance, impedance, or conductance.

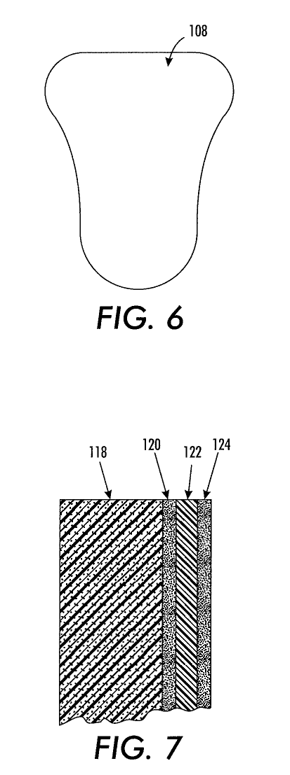

FIGS. 6 and 7 show an example of the sample collection component 108 that may be used in this embodiment. Component 108 may include at least an absorbent material (for example a non-woven cotton material, a porous material, a sponge-like material, etc) with the property of being bibulous (that is, it “drinks up” liquid readily from a source), and the further property of being non-reactive with both the liquid to be collected, the test material, and the patient. The sample collection component 108 is in the form of a liquid collection pad in the illustrated embodiment, which may include an absorbent and bibulous layer 118 of 100% cotton (or other suitable material) held by an adhesive layer 120, which may be made from any commercially available double-sided pressure sensitive adhesive (PSA) suitable for this environment. Adhesive layer 120 also is affixed to a flexible and chemically-inert core layer 122, which may be made from low-density polyethylene (LDPE) or other suitable material, which itself is affixed to another double-sided adhesive layer 124, which may be made from a suitable double-sided PSA. Layer 124 also is used to affix the assembled component 108 to a substrate at the site of liquid collection (in this embodiment, the inside of a diaper). This second, non-allergenic, adhesive layer 124 provides a connection of sufficient strength and resiliency to hold the assembly affixed in the diaper during normal movement and under conditions of temperature and humidity found in a diaper, and may be readily released by a user when the pad is removed from the diaper and placed in the testing location. Layer 124 may also be removably and/or permanently affixed to area 112 of page 101B during testing. Such means may include an adhesive, hook-and-loop and/or other similar means.

A desirable feature of the sample collection component 108, inclusive of layers 118, 120, 122, 124, is that it may be shaped in such a fashion as to adapt to the contours at the site of the liquid collection. In the case in which the liquid to be collected is urine, additional desirable characteristics of the collection component 108 include a physical shape that maximizes the probability of urine collection (as, for example having a larger area in that portion of the pad nearest the urethra) and minimizes the probability of contact with stool or stool water (as, for example, by having a smaller area in that portion of the pad nearest the anal opening). In the case of the collection of liquids from a human source, further desirable features of absorbent layer 118 include construction from medical-grade, hypo- or non-allergenic materials, such as cotton or natural sponge, and a flexible structure that does not produce local pressure points. The sample collection pad may include a multitude of other forms, as disclosed in the prior art, such as U.S. Pat. No. 4,318,984. These forms may include among others felt, porous ceramics or argillaceous materials, glass fibers, wood fibers, cloth, sponge, and polyamide matrices. It will readily be apparent to one skilled in the art that additional features, such as physical barriers, hydrophobic materials, and the like, may also be incorporated into the construction of the pad to further enhance these characteristics.

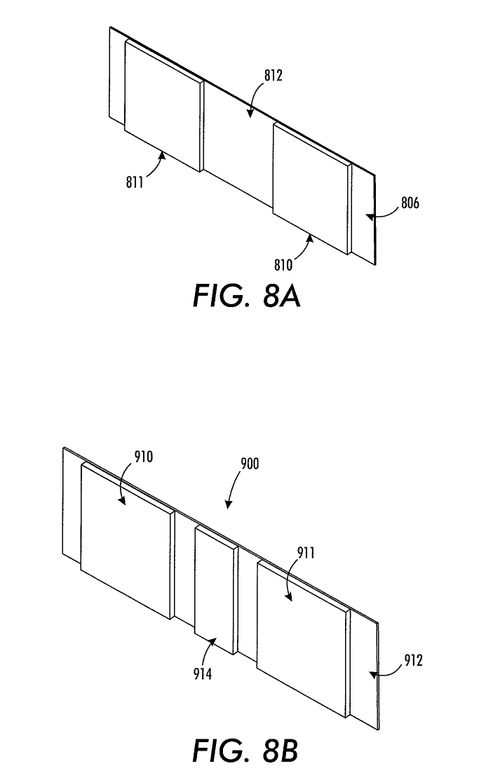

The reagent test strip 806 in FIG. 8A includes two separate reagent pads 810, 811 affixed to an optically-clear backing 812. The reagent pads may be formed from or include suitable materials with sufficient porosity and capillary affinity to cause liquid to migrate into the reagent pad, as is known in the art. The pads also may include suitable reagent-absorbing and/or reagent-adsorbing materials, including but not limited to bibulous materials, such as filter paper, foams, gels, fabrics, phase inversion films as disclosed, e.g. in U.S. Pat. Nos. 4,092,115 and 4,772,561. The optically-clear backing 812 allows a colorimetric result from the reagent pads 810, 811 to be visualized from the side opposite the liquid-contacting side and through the windows 110, 111 in the body 101, as shown in FIGS. 1-5. FIG. 8B shows a dual reagent strip 900 that includes an anti-leaching pad 914 positioned between two reagent strips 910, 911. The anti-leaching pad 914 may also be affixed to an optically-clear backing 912. The pad 914 acts to prevent seepage of liquid from one reagent pad 910 to the other reagent pad 911, because once liquid has interacted with a reagent, the liquid may adversely affect a later reaction with a different reagent.

In use of booklet 100, a user exposes the adhesive layer 124 of the sample collection component 108. The adhesive layer 124 is used to fix the pad in a diaper as close as practicable to the site of urine production. The diaper is then closed and the user awaits urine production. When urine has been produced, the sample collection component 108 holds the sample until such time as the user is able to conduct the test. The user removes the sample collection component 108 from the source and places the liquid-containing pad 108 onto appropriate site 112 on the inner surface 104. The user then closes the booklet 100, which action brings the reagent test strip 106 into direct contact with the liquid collection pad 108, transferring liquid from the collection pad 108 onto the test strip 106. Shown with a transparent cover 101A′, FIG. 4 illustrates the substantial alignment and registration of the reagent strip location 114 and the collection pad location 112, once the body 101 is closed. A sealing means (e.g. any known adhesive strip, snap-lock, etc.) may be provided along the open edge of the inner surfaces 102, 104 of the body 101 to assist in holding the pages closed. The sealing means may also assist in containing any hazardous sample liquid inside of the booklet 100 after use. The user is instructed to press once in the region 114 of the test area to assure good contact between sample collection component 108 and test strip 106. The pressure applied to region 114 expels liquid from sample collection component 108 to the test strip 106. In most embodiments, pressing and holding for several seconds should be adequate to produce consistent results. The user should press long enough to produce an interpretable color change in the reagent strip.

In practice, only a minute amount of liquid is required to activate the test material, as the skilled artisan will appreciate. In one embodiment of a collection pad and reagent test strip constructed according to principles of the invention, a reagent test strip with the dimensions 5 mm²×12 μm thick and capable of imbibing approximately 20 μl of liquid, was sufficiently activated by a sample collection pad holding at least about 100 μl of liquid sample. Accordingly, it is believed that a sample collection pad having at least about 100 μl liquid sample capacity is more than adequate to express at least about 20 μl of sample required to adequately activate the reagent test strip. Of course other amounts may be used depending upon the particular design. For instance, in some designs a collection pad 108 may have a 20 μl liquid sample capacity, which is sufficient to activate a test strip requiring 1 μl of liquid for activation.

Other embodiments may be used to assure good contact between component 108 and reagent pad 106. The device may utilize opposing internal surfaces of the booklet of dissimilar radii of curvature in the open position, such as a flat collection pad surface opposed by a convex reagent pad surface. In this design, closing the booklet “bends” the flat collection pad surface against the curved reagent pad surface and vice-versa, thereby creating a contact pressure that ensures the surfaces come into physical contact. The sealing means ensures that the surfaces remain in contact, thereby eliminating the need for the user to carefully apply a prescribed pressure to the collection pad/reagent pad interface for a critical period of time.

It can be seen that this method and device allows collection of a very small quantity of liquid (e.g. in some embodiments the collection pad may become fully saturated with approximately 1.5 cc of liquid), but still a sufficient quantity to wet the test area with a volume of liquid adequate to trigger and complete the testing. This embodiment therefore overcomes a major obstacle to obtaining urine for testing in infants and other diaper-wearing individuals. An advantage to the user is that the urine collection pad may be set down for a brief period of time before the test is activated, thereby allowing the user to attend to other pressing tasks prior to devoting his or her attention to the test (for example, cleaning and re-diapering the baby in the case of a urine testing embodiment).

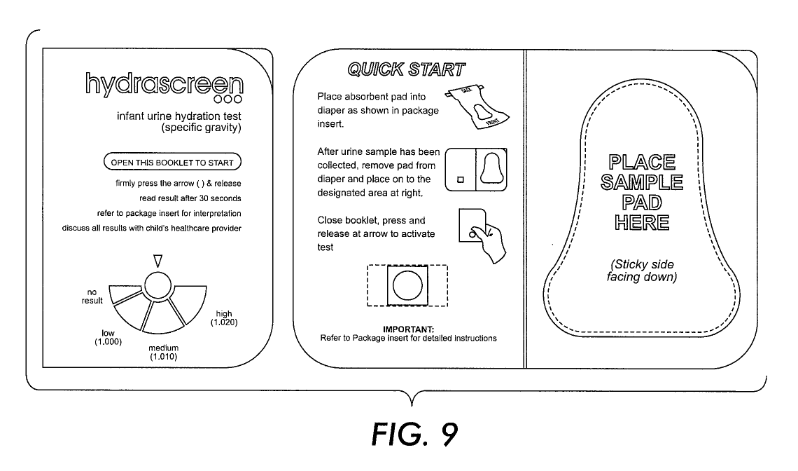

Once the device has been activated by the user (e.g. by closing the booklet as in the above embodiment), the signal produced by the activated test region is visible through the optically-clear windows 110, 111 in the body of the device. After a time period appropriate to the particular test being conducted, the user examines the signal produced by the test area and compares it to the reference standard. In one embodiment, the test signal is a color change, and the reference standard is a reference color chart, for example as illustrated in FIG. 9 discussed below. It will be evident to one skilled in the art that another signal, for example an electronic signal, can be produced as a result of the test interaction, that signal being subsequently processed by electronic or other means and displayed on the body of the device or remotely. It will also be evident that when the signal is a color change, that color change may be read by an automated device that includes a processor in which the test result color characteristics are compared with stored reference color characteristics.

In one embodiment the reference color standard may be incorporated directly into the body of the device, which is an improvement that facilitates ready comparison of the actual test result with the reference color standard. A further desirable feature is that the value or range of values represented by each reference color is displayed immediately adjacent to that reference color. In the case of a binary (yes-no, positive-negative) test that value is displayed adjacent to each reference color, while in the case of a semi-quantitative test in which a range of values is expected, the value or range of values represented by each reference color is displayed adjacent to the appropriate color. An additional desirable feature of the reference color panel is the inclusion of one color that is identical to that of the un-activated test area, in the situation in which the test signal is a color change. This feature provides the user with a “no-test” (also called No Result) indication when insufficient liquid has been delivered to the test area. For example, the color reference chart, as shown in FIG. 9, may illustrate a “No Result” color standard, which may be compared directly with the results from the test. Because the windows 110, 111, as shown in FIGS. 1-4, may be next to or surrounded by the color standard, as shown in FIG. 9 with a single window, the user can easily evaluate the test results and decide if enough liquid has been delivered to the test area and whether the test needs to be repeated. In another embodiment, the device may have more than one test strip and a corresponding number of windows and one or more color reference charts, as shown in FIG. 20 and described in detail below.

An additional desirable feature of the reference color panel, such as shown in FIG. 9, is the asymmetrical arrangement and sizing of the reference color areas—these can be disposed in such a fashion as to make the most concerning color area occupy the largest area, in order to skew the user's subjective assessment towards the abnormal value. This feature is especially desirable in a screening test, and still more desirable in a test intended for use by an untrained user, because it builds in a margin of safety, creating an excess of false-positive interpretations (and thereby minimizing false-negative interpretations, which are the most dangerous in any screening test). An additional margin of safety is provided by the selection of reference colors from the lower end of a continuous scale (or from the negative side of a binary test). This further increases the probability that any error will produce a false-positive, rather than a false-negative, result. For example, as illustrated in FIG. 9, in the case in which the device is a test for urine specific gravity, the reference color indicating “high” is selected from a color chart that illustrates an actual specific gravity of 1.020, a result that to a clinician represents only a moderate elevation of specific gravity (1.030 representing the high end of the actual specific gravity range). It will readily be appreciated that this feature can be applied to tests other than that for specific gravity, and that it has general applicability in guiding a user to the safest decision about any test involving subjective judgment.

It is further desirable that, when the test signal is a color change, the reference colors be developed directly from the actual test material to be used in the device, using externally-validated control solutions to produce each value to be printed in the reference color panel. As will be apparent to one skilled in the art, this can be accomplished by applying a test solution with a known, externally-validated value (as for example, a specific gravity of 1.020 as determined by refractometry) to a certified and validated test strip. At the appropriate time after application of the test liquid, a color-space measurement is made using appropriate equipment. The resulting data provide a reproducible and valid specification for creating the ink used to print each reference color value. By incorporating this feature into the device, a further improvement in accuracy is achieved, because the user is comparing the test area color result with a color that has actually been derived from the test in use. It will be apparent to one skilled in the art that this method is applicable to the development of a reference color panel for any calorimetric test.

An important set of features of the methods and devices disclosed herein includes means for interpretation of a test result and for providing a user with recommendations for actions to be taken in light of the test interpretation. In addition to the features of the test reading panel disclosed above, commercial embodiments may include a detailed “Instructions for Use” package insert that is keyed to the range of results displayed on the body 101 of the device 100 itself. A user conducts the test, reads a result from the range displayed on the body of the device, such as illustrated in FIGS. 9, 14, and 20, and then compares the result with a full interpretation of that result in the package insert. Embodiments of the invention may have instructions on usage and interpretation printed on the device, as illustrated in FIG. 9. In the embodiment in which the device is a means for estimation of urine specific gravity, for example, each color value and numerical result is reproduced on the package insert with specific interpretation of a “low,” “medium,” and “high” result, as shown in FIG. 9. An important feature in this embodiment, which is intended for use in children who may be ill, is a mandatory communication with a child healthcare professional, regardless of the result indicated. This is intended to provide a further margin of safety for the subject of the test. Reproduction of the numerical values associated with each result or range of results is an additional facilitator of communication with the health professional, because it provides results in a fashion familiar to any skilled health professional, and does not require that the professional be familiar with the specific device in use. The interpretation and/or evaluation of the test result may be performed automatically by any device known in the art, such as a computer based refractometer, which eliminates the need for user interpretation/evaluation and may output the result of the test in a suitable display.

Sometimes certain characteristics of a liquid sample may alter the results, or the interpretation of the test results of another sample characteristic. For example, the pH of urine may affect the results of the specific gravity (SG) test of the same urine sample. Therefore, the invention may include two, or more, reagent test strips that may test for more than one characteristic of the sample, such as shown in FIGS. 8A and 8B. The dual windows 110, 111 in page 101A, such as shown in FIGS. 2-4, allow the user to read the results of both tests. The invention may include a key for interpreting the results, in which the result of one test will modulate the result of a second test. For example, a calorimetric interpretation chart may be included with the invention that instructs the user how a pH test result will skew the results of a SG test. For example, as illustrated in FIG. 20, a user is instructed to compare the calorimetric result in test A to the result in test B. Depending on the result in test A, the user is instructed to interpret test B in a certain way, e.g. when A is blue the user must interpret B based on whether B's color falls on either side of a specific line or point. Although FIG. 20 shows interpretation instructions for pH and SG of urine, the invention may be modified for other modular tests for urine and other liquids. The interpretation chart also may be located near the windows 110, 111 on the outside 103 of page 101A, or in another conspicuous location on the invention. The interpretation chart may be adapted for the specific characteristics of the sample being tested using any known means in the art. The invention may be adapted with one or more modular test strips 106, which allows for the testing of one or more different urine conditions at the same time.

The invention also may be embodied to include a pull tab structure to selectively isolate the sample from the testing means. In the embodiments illustrated in FIGS. 10-16, a reagent test strip (specifics are determined by which test is desired) is mounted on a flexible plastic base. The reagent test strip may be constructed and function similar to the reagent test strip 106, as discussed above. Furthermore, the absorbent collection layer may be constructed and function similar to the sample collection component 108 as discussed above. An impermeable plastic shield is interposed between the chemical detection strip(s) and the absorbent collection layer, which is disposed in a position so as to be adjacent to the source of the liquid (e.g., for urine, the patient's urethra). When liquid enters the device it first soaks into the collection layer, where it resides until the test is to commence. No chemicals leach from the test strip while the device is disposed near the patient's skin so long as the impermeable shield remains in place. To commence the test, the user or caregiver removes the device from the patient, and draws out the impermeable shield, which releases the absorbed liquid onto the chemical test strip(s). When timing is necessary, the clock is started at the moment that the impermeable shield is withdrawn. At the conclusion of the requisite time interval, the test strip color is read on the reverse of the device, through the clear plastic that comprises the base of the unit. The appropriate calorimetric legend or scale is disposed adjacent to the test strip(s) for ease of comparison, and where appropriate a “standard” line is disposed on the legend, indicating the value at or above which a concerning result is obtained.

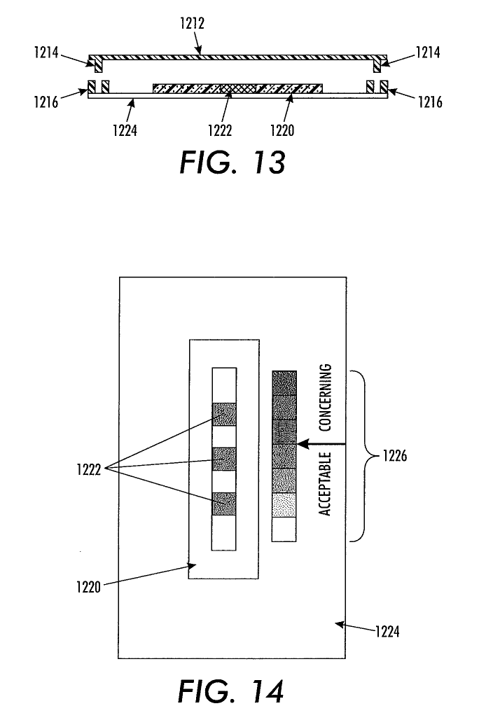

More specifically, FIGS. 10, 11A and 11B illustrate a pull-tab embodiment 1000 that includes a sample collection component in the form of an absorbent collection layer 1010, a liquid-impermeable flexible shield 1012 having a pull tab 1024, a absorbent collection layer 1016, an outer liner 1014, which surrounds and contains the layer 1010, shield 1012, and collection layer 1016, and an adhesive strip 1026 fixed to the bottom of the liner 1014. When in use, the invention is placed inside a patient's diaper so that the absorbent collection layer 1010 faces the wearer's skin. Immediately beneath the absorbent collection layer 1010 is situated the impermeable flexible shield 1012, which is attached at its borders to the outer liner 1014. The shield 1012 prevents the sample liquid from passing from the collection layer 1010 to the test pad 1016. The margin 1024 of the plastic shield 1012 may be scored to allow it to be detached from the outer liner 1014 using firm traction. Beneath the plastic shield 1012 is situated the absorbent collection layer 1016, which is impregnated with the ionic strength indicator solution. The absorbent layer 1010, the plastic shield 1012, and the test strip 1016 are bonded to the outer liner 1014 on three sides 1018. The fourth side 1020 may be attached by a scored margin (not shown) or any releasable connection known in the art, allowing the pull-tab 1022 of the plastic shield 1012 to be withdrawn, breaking the attachment and allowing urine from the absorbent layer 1010 to come into contact with the test strip 1016. A user can gently press the absorbent layer 1010 into the test strip 1016 to urge liquid to contact the test strip 1016. An adhesive strip 1026 is provided to secure the device 1000 to the inside lining of any diaper. The user (or the user's caregiver) can perform the removal of the plastic shield 1012; optionally, the shield 1012 can be removed after removing the entire device 1000 from the wearer's diaper. This method and device allows the timing to start when the user/caregiver is prepared to read the test strip 1016, and also allows the chemicals in the test strip 1016 to be maintained in isolation from the user's skin. The user can control the time of test initiation. After the desired time interval the user or caregiver can read the SG calorimetrically, from the test strip 1016, using the chart provided with the device (e.g. similar to that shown in the embodiment illustrated in FIG. 13.)

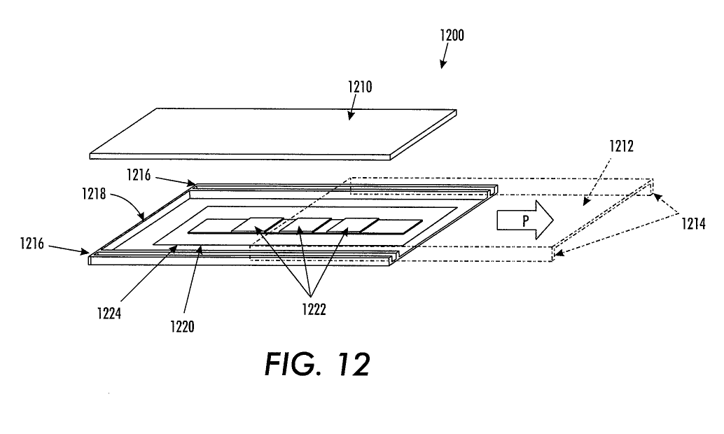

FIGS. 12-14 illustrate a more specific design for carrying out the principles shown in the pull tab embodiment of FIGS. 10-1. FIG. 12 illustrates the use of a “channel lock” method to maintain separation of urine from the reagent test area(s). All of the components described in FIGS. 10-11 are present, with the addition of embossed or otherwise bonded channels running parallel to the long axis of the device along its edges. Zip channels or other releasable sealant means, such as male and female adapters 1214, 1216, prevent leakage from pad 1210 around tab 1212. An absorbent sample collection pad 1210 is separated from the liquid receiving and test pad 1220 by a liquid-impermeable shield 1212, which may be formed from plastic or other suitable material. “Female” (“U”-shaped) adaptors 1216 are bonded to the base 1224 (diaper-side) material of the unit 1200, and “male” (“I”-shaped) adaptors 1214 are bonded to the impermeable plastic shield 1212. The engagement of the male and female adapters 1214 and 1216 provides a secure and watertight bond between the shield 1212 and the base portion 1224 bearing test pad 1220 and the test strips 1222, and also allows ease of withdrawal of the shield 1212 when the test is activated. A third “male” adaptor (not shown) can be disposed at right angles to the others at the end of the impermeable shield 1212, to further isolate the urine receiving and testing pad 1220 from urine contained in the collection pad 1210. Further details of the channel lock construction, the layering of the collection and receiving pads, and the impermeable strip are shown in FIG. 13. FIG. 14 illustrates an exemplary color legend 1226 to assist the user in interpreting the signal from a calorimetric test strip 1222, in a manner as described above.

In this embodiment, liquid enters the device through an orifice 1228 in the outer patient-side liner 1225 and is soaked up by absorbent pad 1210. The orifice 1228 selectively allows liquid to contact the absorbent pad, while restricting access to larger solids and materials that may contaminate the absorbent pad 1210. The orifice 1228 may be any type of selective filter and may be adapted for use in other embodiments to selectively allow seepage of a liquid sample into an absorbent component, while preventing the transfer of excrement or other solids to the absorbent component and/or test strips. Liquid is kept separate from the calorimetric test strip 1222 by an impermeable shield 1212 until use.

When ready for use, a protruding tab portion of shield 1212 is pulled to remove shield 1212, which then is discarded, allowing liquid in pad 1210 to soak into receiving pad 1220. Urine is then distributed from receiving pad 1220 by an optional mesh distributor 1221, which may be employed to uniformly spread the urine into contact with one or more reagent test strips 1222, which are adhered to the plastic base 1224, which may be made of transparent plastic. After any requisite time interval, the color of strip(s) 1222 is read through back of base 1224 and compared with printed color legend 1226 on outside back of base 1224 (shown in FIG. 13).

A fourth embodiment constructed according to the principles of the invention having a pull tab for selectively isolating the sample collection pad and test strip, which is adapted for use for continent users, is shown in FIGS. 15 and 16. The components of FIGS. 12-14 are present and like elements are given the same reference numerals. The bottom half of wand 1230 in FIG. 15 has been removed, as shown in dashed lines, for purposes of illustrating calorimetric test strip 1222. The additional components of this embodiment are disposed on a plastic wand 1230 for use by direct immersion in the liquid of interest (e.g., by placement into a urine stream or into a collection container). In use, the patient places the wand 1230 into a urine stream so that urine may be collected by the orifice 1228 and transferred to the absorbent pad 1210. The shield 1212 isolates the liquid held in the absorbent pad 1210 until the shield 1212 is actuated by a pulling (traction) force. When the shield 1212 is actuated, liquid is admitted first to the receiving pad 1220, where the liquid then flows to the optional porous, liquid bearing media distributor 1221, such as a mesh distributor. The mesh distributor directs the liquid to the reagent test strip 1222. After a time interval (dependent on the test being performed) the user reads the result, such as a calorimetric indicator, on the reverse side of the wand 1230. As in other embodiments, a code or legend may be included on the device.

A fifth embodiment 1700 constructed according to the principles of the invention having an ampule for selectively isolating the sample collection pad and test strip, is illustrated in FIGS. 17 and 18. This embodiment includes, in addition to other features, an absorbent pad 1710, an outer liner 1724, an ampule 1702, an ampule locator 1730, and a testing reagent 1722. The testing reagent 1722 is isolated in an ampule 1702. The testing reagent 1722 may be any testing means known in the art, such as a liquid or dry reagent. As noted above, the ampule may 1702 be a blister packet, capsule, closed packet, compartment, container, balloon or other appropriate device. The ampule 1702 may be constructed of any appropriate material for a given testing medium including plastic, glass, rubber, polymer, cloth, paper product, or other material. The liner 1724 may support the ampule 1702, beneath the absorbent pad 1710, so that the reagent 1722 may only flow towards the pad 1710. The ampule locator may be a bump or other palpable indicator of the location of the ampule 1702. After urine is collected on the absorbent pad 1710, the user applies force to the ampule locator 1730 causing the ampule 1702 to rupture. The reagent 1722 emerges from the ampule 1702 and interacts with the urine in the absorbent pad 1710. The results can be interpreted in accordance with the type of test and reagent used. For example, the reagent may be an ionic strength indicator and interact with urine collected in the absorbent pad 1710 and turn the absorbent pad 1710 a color indicating the specific gravity (SG) of the urine. The color can then be compared with the color chart, similar to FIG. 14, provided with the device.

A sixth embodiment constructed according to the principles of the invention includes a diaper hydration pad 1900 having a sample collection pad 1910, a reagent test strip 1930 integrated into a liquid test pad 1916, and an outer liner 1914, as shown in FIG. 19. In this embodiment, the sample collection pad 1910 includes both the liquid test strip 1916 and the reagent test strip 1930. The diaper pad 1900 has an impermeable outer liner 1914, which supports a liquid collection pad 1910 and the liquid test pad 1916. The outer liner 1914 can be shaped with curved edges 1918 to more appropriately match the shape of the inside of a diaper. The curved edges 1918 can fit the leg recesses in a diaper without bunching up; this improves the comfort for the wearer. The straight edges 1920 of the hydration pad extend 1900 anteriorly and posteriorly of the patient to increase the coverage and sample collection ability. For similar reasons, the margins 1924 of the liquid collection pad 1910 extend close the edges of the outer liner 1914. The embedded reagents 1930 are illustrated in positions which closely correspond to the anatomical location of the female urethra, but can be positioned in a diaper suitable for collection from a male patient. For additional embodiments, the reagent strip can be located at any position in the liquid test pad 1916. The reagents 1930 may be isolated from the patient's skin as well as the liquid collection pad 1910 by a removable plastic shield, which a user removes after the hydration pad 1900 has been removed from the diaper. After the shield has been removed, the urine can distribute, via capillary action, from the collection pad 1910 to the liquid test pad 1916 and interact with the reagents 1930. The user can then interpret the results, calorimetric or other, with an interpretation guide, as illustrated in FIG. 14. This embodiment also may include more than one modular reagents 1930, which may test multiple characteristics of a sample or one reagent may modify the results of another reagent. If modifying reagents are used, then an interpretation guide like that shown in FIG. 20 may be used.

In addition to determining the hydration status of a patient, embodiments of the invention may be adapted to test for the presence, absence, or relative level of at least the following substances:

In Urine, analytes commonly tested in urinalysis, markers for in-born errors of metabolism, metabolites of prescribed or abused drugs, including but not limited to:
Glucose
Protein
Bilirubin
Urobilinogen
Red blood cells
Creatinine
Specific gravity (SG)
pH
Leukocyte esterase (presence of white blood cells)
Nitrites (metabolic products of reproducing bacteria)
Ketones
Human Chorionic Gonadotropin (evidence of pregnancy and/or ovulation)
Various drugs and their metabolites
Amino acids and other organic acids
Sexually Transmitted Infections (STI)
Metabolic products of oxidant damage to tissue (e.g., thiobarbituric acid reducing substances, or TBARS)
Presence of antigens from a large number of bacterial organisms that cause human disease
Presence of antigens from a large variety of parasitic organisms, most immediately bladder-dwelling parasites such as Schistosoma hematobium, the leading cause of bladder cancer in the world
Oxalic acid (a major cause of kidney and bladder stones)
Genetic material such as DNA and/or RNA sequences, or fragments thereof.

In saliva, including but not limited to:
Theophylline and other therapeutic drugs
ortisol and other endogenous steroids
Hydration
Genetic material such as DNA and/or RNA sequences, or fragments thereof.

Thus, the invention can take a form and method which is desired to conduct a given test on a given liquid. The most advantageous embodiments of the invention provide for the collection and isolation of liquid, transportation or contact of liquid with a testing medium and the testing of liquid. A number of embodiments of this invention allow for each of these steps to be conducted in the same device in a convenient manner which is comfortable to the patient and in which the timing of the test is under the control and choosing of the one conducting the test.

The invention may also contain additional operational features that build on the non-invasive sample collection and analysis as described in the above embodiments. The invention may be adapted to have a multiplexing capability, which may include the use of multiple test strips, with multiple reagents, or other means for splitting the sample to permit multiple assays in a single booklet or other embodiment of the invention. For example, as discussed above, the invention may include two or more reagent test strips, each testing a different characteristic of the sample, such as pH and SG of a urine sample or the testing for the presence of a drug or steroid, and/or hydration of a saliva sample. One test result may be used to assist in the interpretation of another test result, such as pH and SG, or the multiple reagent test strips may be interpreted independently without regard to another result. Thus, embodiments of the invention, particularly the booklet embodiments, as shown in FIGS. 1-4, may be modular in form to provide testing and reading of multiple reagent test strips, such as having a number of windows 110, 111 corresponding to the number of test strips. The booklet 100 is illustrated with two windows 110, 111, but it is within the scope of the invention to have any number of test strips and corresponding windows, as well as means for interpreting the test results. Other embodiments of the invention also may be adapted to include multiple test strips for testing multiple characteristics of a sample liquid and preparation of the sample for testing, such as described previously.

The invention may also have means for archiving samples and/or analysis results, as well as, providing positive sample identification (PSID), which creates a link between patients' sample and a test result and reduces laboratory errors. Archiving samples may help prevent mixing up of patient results and PSID may provide for later use of the sample or test device.

The invention may also be adapted to integrate the collection means and the testing means to an automated testing or diagnostic instrument. The sample collection means embodied in the invention may be transposable to a testing instrument to deliver the sample to the test instrument for substitute or additional tests. For example, the sample collection means may be integrated into other diagnostic methodologies, particularly molecular diagnostic assays that utilize DNA amplification technology, such as polymerase chain reaction (PCR). Additionally, the testing means embodied in the invention may be adapted to be interpreted by secondary mechanical means by using calorimetric dyes of other non-visible dyes.

The sample collection component of the invention may have other activation modes. For example, it may serve as a liquid bridge connecting two electrical half-cells, which may then be activated by bringing electrically conductive probes into contact with each half-cell to perform electro-analytical measurements of the sample, such as current or potential. The invention, the sample collection component may be placed into its designated position and the device may be activated by directing electromagnetic radiation at the wetted component to perform electromagnetic measurements such as transmission, dispersion, refraction, reflection, scattering, polarization, photoelectric, absorption, relaxation, or emission.

The test medium of the invention may take multiple forms, such as reagent strips or chip-based detectors that may produce an electrochemical result. Other embodiments of the invention may be adapted to filter a collected sample before testing it. Biological samples frequently contain material other than the analyte of interest, and certain materials may interfere with proper performance of the test being conducted. As will be apparent to one skilled in the art, it is possible to construct an analysis element such as a chemical reagent strip, biosensor, or other component, in such a fashion as to include a filter, an adsorbent layer, or another means of effecting separation of desired from undesirable or interfering materials. Incorporation of such an analysis element has the desirable effect of allowing delivery of a substantially purified, concentrated, or otherwise enhanced liquid to the final site of analysis. An additional beneficial effect is the removal of materials that, while not interfering with the analytical process itself, might interfere with interpretation of the signal indicating the result—for example, even an inert colored material could interfere with the color signal produced by a calorimetric chemical test strip.

In summary, we have disclosed one or more methods and devices that permit the collection of very small quantities of liquid from a liquid source that need not actively participate in the liquid collection. We have provided a means of maximizing the probability of ample liquid collection while minimizing the probability of contamination or adulteration of the liquid specimen. We have provided a means of assuring adequate fixation of the liquid collection means in relationship to the source of the liquid, while allowing ready removal of the liquid collection means to the specimen testing area. We have disclosed means of registering the liquid collection means with the liquid testing means in such a way as to require minimal skills of a user, so as to maximize the probability of delivering analyzable sample to the test area. We have provided a means of making the test result signal evident to a user and for facilitating the reading of the test result. We have provided a series of means of providing various margins of safety into the process of subjectively reading a result when the result is a calorimetric comparison with a set of reference colors, and we have provided means of assuring the accuracy and reproducibility of the reference colors. We have further provided means that facilitate interpretation of test results and of communicating those results with a person having a higher level of knowledge and skills than those of the person conducting the test, and we have provided means of acting on the results of the test so as to speed mitigation of a problem as detected by the test.

The description and examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention, which are obvious to those skilled in the medical sciences, urology, pediatrics, or related fields are intended to be within the scope of the appended claims.

The disclosures of all references and publications cited above are expressly incorporated by reference in their entireties to the same extent as if each were incorporated by reference individually.

What is claimed is:

1. A process for the analysis of a biological liquid, said process comprising the steps of:
collecting a first volume of biological liquid from its source without the use of instrumentation and without the direct participation, manipulation, or modification of the source;
directly transferring a minimum quantity of a second volume from the collected volume to an analysis system sufficient to activate the analysis system at a location remote from the source without intervening manipulation of the second volume; and
producing a signal containing information about at least one property of the biological liquid.

2. The process of claim 1, further comprising evaluating the signal.

3. The process of claim 2, wherein said evaluating step comprises evaluating the signal by a user not specifically trained in the analysis being conducted.

4. The process of claim 1, wherein said liquid being tested is a human body liquid and the first volume is at least about 20 μl, and the second volume is at least about 1 μl.

5. The process of claim 4, wherein said human body liquid is one of urine, saliva, blood, sweat, tears, plasma, serum, milk, spinal fluid, lymph fluid, secretions from the respiratory tract, secretions from the intestinal tract, and secretions from the genitourinary tract.

6. The process of claim 1, wherein said collecting step comprises isolating the first volume from the analysis system.

7. The process of claim 1, wherein said transferring step includes contacting the second volume with at least one chemically, biologically, biochemically, or electrochemically reactive analysis element after a time period selected by the user expires.

8. A method for testing liquids obtained non-invasively from an animal or human subject, said method comprising the steps of:
positioning a collecting device in a desired location relative to the subject;
collecting a liquid sample from the subject in the collecting device while it is in its desired location relative to the subject;
supporting the collecting device in a fixed special relationship to an analysis element;
registering the collecting device with the analysis element; and
transferring liquid from the collecting device to the analysis element to enable a reaction to occur between the liquid and the analysis element, with the reaction being indicative of a characteristic of the liquid.

9. The method of claim 8, comprising evaluating the results of the analysis based upon the characteristic to indicate a condition of the subject.

10. The method of claim 8, further including the step for transporting the collecting device to a location in proximity to the analysis element.

11. The method of claim 10, wherein said transporting step is accomplished by a user without specific training, permits preservation of the integrity of the sample, and obviates the need for any further processing prior to being transported to a location in proximity to the analysis element.

12. The method of claim 8, wherein said steps occur in a support body providing a user with control over the timing and location of each of said steps in the testing process.

13. The method of claim 8, wherein said registering step includes the use of instructions included with the support body that describe graphically and verbally how to properly register the collection device and the analysis element such that proper registration is evident to a user without specific training.

14. The method of claim 8, wherein said supporting step prevents premature interaction of liquid in the collecting device and the analysis element.

15. The method of claim 8, further comprising introducing at least one discrete and variable time interval between said transporting and registration steps such that activation of the analysis element occurs without substantial loss of function of the test or its validity.

16. The method of claim 8, wherein said supporting step comprises permanently attaching the collection device to a support body including the analysis element to facilitate subsequent easy disposal of the collection device and analysis element.

17. The method of claim 8, wherein said supporting step comprises removably attaching the collection device to a support body including the analysis element such that the collection device may be removed from the support body following said transferring step, thereby allowing the collection device to be safely transported to another location without the support body.

18. The method of claim 8, wherein the analysis element does not contact the subject in any of the steps to reduce risk of an adverse reaction between the materials used in the analysis element and the subject.

19. The method of claim 8, wherein said transferring step comprises one of i) directly contacting the analysis element with the collecting device; ii) removing a separator between the analysis element and the collection device, and iii) breaking an ampule.
